(19) United States
(12) Patent Application Publication
(10) Pub. No.: US 2012/0015919 A1
(43) Pub. Date: Jan. 19, 2012

(54) THERAPIES FOR TREATING CANCER USING COMBINATIONS OF COX-2 INHIBITORS AND AROMATASE INHIBITORS OR COMBINATIONS OF COX-2 INHIBITORS AND ESTROGEN RECEPTOR ANTAGONISTS

(75) Inventors: Thomas M. Estok, Carlsbad, CA (US); Sara L. Zaknoen, Carlsbad, CA (US); Robert K. Mansfield, Carlsbad, CA (US); Tracy Lawhon, Encinitas, CA (US)

(73) Assignee: Tragara Pharmaceuticals, Inc., San Diego, CA (US)

(21) Appl. No.: 13/132,893

(22) PCT Filed: Sep. 23, 2008
(86) PCT No.: PCT/US08/77401

§ 371 Date: Sep. 28, 2011

Related U.S. Application Data
(60) Provisional application No. 60/974,746, filed on Sep. 24, 2007.

(51) Int. Cl. A61K 31/5685; A61K 31/4196; A61P 35/00; A61K 31/402

(52) U.S. Cl. 514/171; 514/427; 514/383

(57) ABSTRACT

Described herein are compositions and methods for using these compositions in the treatment of cancer, tumors, and tumor-related disorders in a subject.

### CROSS-REFERENCE

This application claims the benefit of U.S. Provisional Application No. 60/974,746, filed Sep. 24, 2007, which is incorporated herein by reference in its entirety.

### FIELD

The present invention relates to combination compositions and the use of such combinations for the treatment of cancer, tumors, and tumor-related disorders.

### BACKGROUND

Cancer, tumors, tumor-related disorders, and neoplastic disease states are serious and often times life-threatening conditions. These diseases and disorders, which are characterized by rapidly-proliferating cell growth, continue to be the subject of research efforts directed toward the identification of therapeutic agents which are effective in the treatment thereof. Such agents prolong the survival of the patient, inhibit the rapidly-proliferating cell growth associated with the neoplasm, or effect a regression of the neoplasm.

Generally, surgery and radiation therapy are the first modalities considered for the treatment of cancer that is considered locally confined, and offer the best prognosis. Chemotherapy treatment of certain cancers typically results in disappointing survival rates but still offer a survival benefit. For example, in patients with breast cancer, aromatase inhibitor chemotherapy regimens, such as the use of letrozole, anastrozole or exemestane, are employed. If patients fail to respond to an aromatase inhibitor treatment, additional conventional treatment offers limited benefit.

Despite the approval of several aromatase inhibitors for the treatment of early and late stage breast cancer, as with most therapeutic agents, side-effects result from its use. For example, common side effects include hot flashes, vasodilation and nausea. Of greater concern, is the growing view that, while utilization of aromatase inhibitors for the treatment of tumors may initially shrink the size of the tumor, the tumor may eventually enlarge in size, indicating, among other things, the development of resistance. Letrozole, a widely used aromatase inhibitor, may be representative of the types of therapeutic agents being used for cancer treatment; in that its use has an effect on cancer, but because of other factors, which are not entirely known, the tumor develops resistance and progresses.

What is needed, therefore, are compositions and/or methods of treatment for cancer which take advantage of the synergy found in a therapeutic combination that could increase the effectiveness of the agents and reduce and/or eliminate the side effects typically associated with conventional treatments.

### SUMMARY OF THE INVENTION

### Methods of Use

Provided herein are methods of treating cancer based on the administration of a combination of a 1,2-diphenylpyrrole derivative (a COX-2 selective inhibitor) and an aromatase inhibitor. The methods may further include treatments wherein the combination is supplemented with one or more therapeutic agents or therapies. The 1,2-diphenylpyrrole derivative and the aromatase inhibitor may be provided in separate dosage forms or combined in one dosage form (e.g. a fixed dose).

In another embodiment, the invention provides a composition comprising a combination of a 1,2-diphenylpyrrole derivative and an aromatase inhibitor wherein the 1,2-diphenylpyrrole derivative is 2-(4-ethoxyphenyl)-4-methyl-1-(4-sulfamoylphenyl)-pyrrole and wherein the NSAID-induced side effects are substantially diminished.

Also provided herein are methods of treating cancer based on the administration of a combination of a 1,2-diphenylpyrrole derivative and an estrogen receptor antagonist. The methods may further include treatments wherein the combination is supplemented with one or more therapeutic agents or therapies. The 1,2-diphenylpyrrole derivative and the estrogen receptor antagonist may be provided in separate dosage forms or combined in one dosage form (e.g. a fixed dose).

In another embodiment, the invention provides a composition comprising a combination of a 1,2-diphenylpyrrole derivative and an estrogen receptor antagonist wherein the 1,2-diphenylpyrrole derivative is 2-(4-ethoxyphenyl)-4-methyl-1-(4-sulfamoylphenyl)-pyrrole and wherein the NSAID-induced side effects are substantially diminished.

1,2-Diphenylpyrrole derivatives described herein have the general formula:

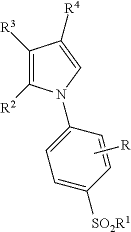

wherein:

R is a hydrogen atom, a halogen atom or an alkyl group having from 1 to 6 carbon atoms;

R¹ is an alkyl group having from 1 to 6 carbon atoms or an amino group;

R² is a phenyl group which is unsubstituted or is substituted by at least one substituent selected from the group consisting of substituents a and substituents β;

R³ is a hydrogen atom, a halogen atom or an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms;

R⁴ is a hydrogen atom; an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms; a cycloalkyl group having from 3 to 8 carbon atoms, an aryl group; or an aralkyl group; said aryl group having from 6 to 14 ring carbon atoms in a carbocyclic ring and are unsubstituted or are substituted by at least one substituent selected from the group consisting of substituents a and substituents β; said aralkyl group are an alkyl group having from 1 to 6 carbon atoms and which are substituted by at least one aryl group as defined above;

said substituents a are selected from the group consisting of a hydroxy group, a halogen atom, an alkoxy group having from 1 to 6 carbon atoms and an alkylthio group having from 1 to 6 carbon atoms;

said substituents β are selected from the group consisting of an alkyl group which has from 1 to 6 carbon atoms and which is unsubstituted or are substituted by at least one substituent selected from the group consisting of a hydroxy group, a halogen atom, an alkoxy group having from 1 to 6 carbon atoms and an alkylthio group having from 1 to 6 carbon atoms; an alkanoyloxy group having from 1 to 6 carbon atoms; a mercapto group; an alkanoylthio group having from 1 to 6 carbon atoms; an alkylsulfinyl group having from 1 to 6 carbon atoms; a cycloalkloxy group having from 3 to 8 carbon atoms; a haloalkoxy group having from 1 to 6 carbon atoms; and an alkylenedioxy group having from 1 to 6 carbon atoms; or a pharmaceutically acceptable salt, solvate, or prodrug.

In one embodiment, the invention provides a 1,2-diphenylpyrrole derivative having the formula:

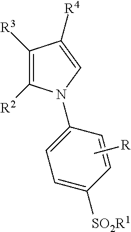

wherein:

R is a hydrogen atom, a halogen atom or an alkyl group having from 1 to 4 carbon atoms;

R¹ is a methyl group or an amino group;

R² is an unsubstituted phenyl group or a phenyl group which is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and which is substituted by at least one substituent selected from the group consisting of a halogen atom, an alkoxy group having from 1 to 4 carbon atoms and an alkylthio group having from 1 to 4 carbon atoms; a haloalkoxy group having from 1 to 4 carbon atoms; and an alkylenedioxy group having from 1 to 4 carbon atoms;

R³ is a hydrogen atom, a halogen atom, an unsubstituted alkyl group having from 1 to 4 carbon atoms or a substituted alkyl group having from 1 to 4 carbon atoms and substituted by at least one substituent selected from the group consisting of a halogen atom, an alkoxy group having from 1 to 4 carbon atoms and an alkylthio group having from 1 to 4 carbon atoms;

R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to carbon atoms; a cycloalkyl group having from 3 to 6 carbon atoms; an aryl group which has from 6 to 10 ring carbon atoms and which is unsubstituted or is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to 4 carbon atoms; and a cycloalkyloxy group having from 3 to 7 carbon atoms; an aralkyl group having from 1 to 4 carbon atoms in the alkyl part and containing at least one said aryl group; or a pharmaceutically acceptable salt, solvate, or prodrug.

In one embodiment, the invention provides a 1,2-diphenylpyrrole derivative wherein:

R is a hydrogen atom;

R¹ is an amino group;

R² is an unsubstituted phenyl group or a phenyl group which is substituted by at least one substituent selected from the group consisting of a halogen atom, an alkoxy group having from 1 to 4 carbon atoms, an alkylthio group having from 1 to 4 carbon atoms, an alkyl group having from 1 to 4 carbon atoms, a haloalkyl group having from 1 to 4 carbon atoms, a haloalkoxy group having from 1 to 4 carbon atoms and a alkylenedioxy group having from 1 to 4 carbon atoms;

R³ is a hydrogen atom, a halogen atom, an alkyl group having from 1 to 4 carbon atoms or a haloalkyl group having from 1 to 4 carbon atoms;

R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group and an alkoxy group having from 1 to 4 carbon atoms; a cycloalkyl group having from 3 to 6 carbon atoms; an aryl group which has from 6 to 10 ring carbon atoms and which is unsubstituted or is substituted by at least one substituent selected from the group consisting of a hydroxy group; a halogen atom; an alkoxy group having from 1 to 4 carbon atoms; an unsubstituted alkyl group having from 1 to 4 carbon atoms; an alkyl group having from 1 to 4 carbon atoms and which is unsubstituted or substituted by at least one halogen atom; and a cycloalkyloxy group having from 3 to 7 carbon atoms; and an aralkyl group having from 1 to 4 carbon atoms in the alkyl part and containing at least one said aryl group; or a pharmaceutically acceptable salt, solvate, or prodrug.

In one embodiment, R is a hydrogen atom. In another embodiment, R is a fluorine atom. In a further embodiment, R is a chlorine atom. In yet a further embodiment, R is a methyl group.

In one embodiment, R¹ is a methyl group. In another embodiment, R¹ is an amino group.

In one embodiment, R² is a phenyl group.

In one embodiment, R³ is a hydrogen atom. In another embodiment, R³ is a halogen atom.

In one embodiment, R⁴ is a hydrogen atom.

The term “aryl” refers to a carbocyclic aromatic hydrocarbon group having from 6 to 14 carbon atoms in one or more aromatic rings or such a group which is fused to a cycloalkyl group having from 3 to 10 carbon atoms, and the group is unsubstituted or it is substituted by at least one substituent selected from the group consisting of hydroxy groups, halogen atoms, lower alkoxy groups, lower alkylthio groups, lower alkyl groups, alkanoyloxy groups, mercapto groups, alknoylthio groups, lower alkylsulfinyl groups, lower alkyl groups having at least one substituent selected from the group consisting of cycloalkloxy groups, lower haloalkoxy groups, and lower alkylenedioxy groups.

In some embodiments, the 1,2-diphenylpyrrole derivative is selected from the group consisting of compounds 2-1-2-213 of Table 2 as disclosed in U.S. Pat. No. 6,887,893, which is herein incorporated in its entirety by reference.

In one embodiment, the 1,2-diphenylpyrrole derivative is selected from the group consisting of: 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole. In another embodiment, the invention provides a method wherein the 1,2-diphenylpyrrole derivative is 2-(4-ethoxyphenyl)-4-methyl-1-(4-sulfamoylphenyl)-pyrrole.

In another embodiment, the 1,2-diphenylpyrrole derivative is 2-(4-ethoxyphenyl)-4-methyl-1-(4-sulfamoylphenyl)-pyrrole.

The methods for synthesizing 1,2-diphenylpyrrole derivatives, illustrated herein, are described in the Examples section and in U.S. RE39,420, which is incorporated herein by reference in its entirety.

In another embodiment the aromatase inhibitor is letrozole.

In a further embodiment, the 1,2-diphenylpyrrole derivative is 2-(4-ethoxyphenyl)-4-methyl-1-(4-sulfamoylphenyl)-pyrrole and the aromatase inhibitor is letrozole.

In another embodiment the aromatase inhibitor is anastrozole.

In a further embodiment, the 1,2-diphenylpyrrole derivative is 2-(4-ethoxyphenyl)-4-methyl-1-(4-sulfamoylphenyl)-pyrrole and the aromatase inhibitor is anastrozole.

In another embodiment the aromatase inhibitor is exemestane.

In a further embodiment, the 1,2-diphenylpyrrole derivative is 2-(4-ethoxyphenyl)-4-methyl-1-(4-sulfamoylphenyl)-pyrrole and the aromatase inhibitor is exemestane.

In yet a further embodiment the 1,2-diphenylpyrrole derivative and the aromatase inhibitor are administered sequentially in either order or simultaneously In another embodiment the 1,2-diphenylpyrrole derivative and the estrogen receptor antagonist are administered sequentially in either order or simultaneously.

In one embodiment the 1,2-diphenylpyrrole derivative is administered first.

In another embodiment the aromatase inhibitor is administered first.

In another embodiment the estrogen receptor antagonist is administered first.

In another embodiment, the present invention provides a method for treating a subject having cancer, comprising administering to the subject, a therapeutically effective amount of a combination comprising a 1,2-diphenylpyrrole derivative and an estrogen receptor antagonist or their respective pharmaceutically acceptable salts, solvates, polymorphs or prodrugs.

Additionally, the present invention provides a method for treating a subject having cancer, comprising administering to the subject, a therapeutically effective amount of a combination comprising a 1,2-diphenylpyrrole derivative and an estrogen receptor antagonist or their respective pharmaceutically acceptable salts, solvates, polymorphs or prodrugs wherein the 1,2-diphenylpyrrole derivative is 2-(4-ethoxyphenyl)-4-methyl-1-(4-sulfamoylphenyl)-pyrrole and the estrogen receptor antagonist is selected from fulvestrant, tormifene, raloxifene and tamoxifen.

In another embodiment the estrogen receptor antagonist is fulvestrant.

In another embodiment the 1,2-diphenylpyrrole derivative is 2-(4-ethoxyphenyl)-4-methyl-1-(4-sulfamoylphenyl)-pyrrole and the estrogen receptor antagonist is fulvestrant.

In another embodiment the estrogen receptor antagonist is tamoxifen.

In another embodiment the 1,2-diphenylpyrrole derivative is 2-(4-ethoxyphenyl)-4-methyl-1-(4-sulfamoylphenyl)-pyrrole and the estrogen receptor antagonist is tamoxifen.

In another embodiment the estrogen receptor antagonist is toremifene.

In another embodiment the 1,2-diphenylpyrrole derivative is 2-(4-ethoxyphenyl)-4-methyl-1-(4-sulfamoylphenyl)-pyrrole and the estrogen receptor antagonist is toremifene.

In another embodiment the estrogen receptor antagonist is raloxifene.

In another embodiment the 1,2-diphenylpyrrole derivative is 2-(4-ethoxyphenyl)-4-methyl-1-(4-sulfamoylphenyl)-pyrrole and the estrogen receptor antagonist is raloxifene.

In one embodiment, the invention provides a method for treating cancer, tumors, and tumor-related disorders, comprising administering the combination by a mode of administration comprising oral, parenteral, buccal, intranasal, epidural, sublingual, pulmonary, local, rectal, or transdermal administration.

In one embodiment, the invention provides a method for treating cancer, tumors, and tumor-related disorders wherein the combination is orally administered as a single dosage form.

In another embodiment, the invention provides a method for treating cancer, tumors, and tumor-related disorders wherein the single dosage form enhances patient compliance and/or reduces pill burden.

In a further embodiment, the invention provides a method for treating cancer, tumors, and tumor-related disorders wherein the single dosage form is a single capsule or a single tablet.

In yet a further embodiment, the invention provides a method for treating cancer, tumors, and tumor-related disorders wherein the composition is provided as a single tablet.

In a further embodiment, the invention provides a method for treating cancer, tumors, and tumor-related disorders, comprising administering the combination by a mode of parenteral administration selected from intravenous, subcutaneous, intrathecal, and intramuscular administration.

### Dosages

In a further embodiment, the invention provides a method wherein the aromatase inhibitor is a small molecule compound. In one embodiment, the invention provides a method wherein the small molecule compound is selected from the group consisting of: letrozole, anastrozole and exemestane.

In another embodiment, the invention provides a method wherein the estrogen receptor antagonist is a small molecule compound. In one embodiment, the invention provides a method wherein the small molecule compound is selected from the group consisting of: tamoxifen, toremifene, raloxifene and fulvestrant.

In one embodiment, the invention provides a method for treating cancer, tumors, and tumor-related disorders comprising administering the combination in a single tablet wherein the single tablet comprises from about 100 mg to about 1200 mg of 2-(4-ethoxyphenyl)-4-methyl-1-(4-sulfamoylphenyl)-pyrrole and from about 1.0 mg to about 3.0 mg of letrozole.

In another embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 1.0 mg of letrozole.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 1.5 mg of letrozole.

In yet a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 2.0 mg of letrozole.

In another embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 2.5 mg of letrozole.

In yet another embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 3.0 mg of letrozole.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and from about 0.5 mg to about 1.5 mg of anastrozole.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 0.5 mg of anastrozole.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 1.0 mg of anastrozole.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 1.5 mg of anastrozole.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and from about 5 mg to about 35 mg of exemestane.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 5 mg of exemestane.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 12 mg of exemestane.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 25 mg of exemestane.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 35 mg of exemestane.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and from about 10 mg to about 50 mg of tamoxifen.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 10 mg of tamoxifen.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 20 mg of tamoxifen.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 30 mg of tamoxifen.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 40 mg of tamoxifen.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 50 mg of tamoxifen.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and from about 30 mg to about 75 mg of toremifene.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 30 mg of toremifene.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 45 mg of toremifene.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 60 mg of toremifene.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 75 mg of toremifene.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and from about 30 mg to about 60 mg of raloxifene.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 30 mg of raloxifene.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 40 mg of raloxifene.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 50 mg of raloxifene.

In a further embodiment, the invention provides a method for treating cancer, tumors, and tumor-related disorders wherein the single tablet comprises from about 100 mg to about 1200 mg of 2-(4-ethoxyphenyl)-4-methyl-1-(4-sulfamoylphenyl)-pyrrole and about 60 mg of raloxifene.

In one embodiment, the invention provides a method of treating cancer, tumors, and tumor-related disorders comprising administering a composition comprising a combination of a 1,2-diphenylpyrrole derivative and an aromatase inhibitor wherein the composition is suitable for once-daily administration.

In one embodiment, the invention provides a method of treating cancer, tumors, and tumor-related disorders comprising administering a composition comprising a combination of a 1,2-diphenylpyrrole derivative and an estrogen receptor antagonist wherein the composition is suitable for once-daily administration.

In a further embodiment, the invention provides a method wherein administering the combination enhances treatment of the subject compared to administering one component of the combination alone.

In yet a further embodiment, the invention provides a method wherein administering the combination reduces the side effects of treatment for a cancer, tumor, or a tumor-related disorder.

### Further Methods of Use

In one embodiment, the invention provides a method of inducing differentiation of tumor cells, the method comprising contacting the cells with an effective amount of a combination comprising a 1,2-diphenylpyrrole derivative and an aromatase inhibitor whereby the combination induces differentiation of tumor cells.

In one embodiment, the invention provides a method of inhibiting proliferation of cancer cells, the method comprising contacting a cancer cell with a combination comprising a 1,2-diphenylpyrrole derivative and an aromatase inhibitor whereby the combination inhibits proliferation of cancer cells.

In another embodiment, the invention provides a method for reducing proliferation of cancer cells, the method comprising delivering to the cells a combination comprising a 1,2-diphenylpyrrole derivative and an aromatase inhibitor, whereby the reduction of cell proliferation is greater than a reduction caused by either a 1,2-diphenylpyrrole derivative alone or an aromatase inhibitor alone.

In one embodiment, the invention provides a method of modulating the immune response, the method comprising delivering to a cancer cell an effective amount of a combination comprising a 1,2-diphenylpyrrole derivative and an aromatase inhibitor wherein the combination modulates the immune response.

In a further embodiment, the invention provides a method of inhibiting metastases of tumor cells, the method comprising administering an effective amount of a combination comprising a 1,2-diphenylpyrrole derivative and an aromatase inhibitor such that the combination inhibits metastatic activity of tumor cells.

In one embodiment, the invention provides a method for inducing apoptosis in cancer cells, the method comprising contacting the cancer cells with a combination comprising a 1,2-diphenylpyrrole derivative and an aromatase kinase sufficient to induce apoptosis.

In another embodiment, the invention provides a method for sensitizing aromatase inhibitor resistant cancer cells to an aromatase inhibitor, the method comprising administering a combination comprising a 1,2-diphenylpyrrole derivative and an aromatase inhibitor wherein the combination sensitizes the cancer cells to the aromatase inhibitor.

In one embodiment, the invention provides a method of treating aromatase resistance in a cancer cell, the method comprising, administering a combination comprising a 1,2-diphenylpyrrole derivative and an aromatase inhibitor.

In one embodiment, the invention provides a method of treating resistance to an inhibitor of ErbB1 and/or ErbB2 in a cancer cell, the method comprising, administering a combination comprising a 1,2-diphenylpyrrole derivative and an aromatase inhibitor in combination with an inhibitor of ErbB1 and/or ErbB2.

In a further embodiment, the invention provides a method of modulating prostaglandin synthesis in a cancer cell, the method comprising contacting the cell with a combination comprising a 1,2-diphenylpyrrole derivative and an aromatase inhibitor wherein the combination inhibits prostaglandin synthesis in a cancer cell.

In one embodiment, the invention provides a method of modulating cyclooxygenase expression in a cancer cell, the method comprising delivering to the cell a combination comprising a 1,2-diphenylpyrrole derivative and an aromatase inhibitor wherein the combination inhibits cyclooxygenase expression in a cancer cell.

In one embodiment, the invention provides a method of modulating angiogenesis in a cancer cell, the method comprising contacting the cell with a combination comprising a 1,2-diphenylpyrrole derivative and an aromatase inhibitor wherein the combination inhibits angiogenesis in a cancer cell.

In another embodiment, the invention provides a method of reducing the dosage in conventional treatment for neoplasia and/or neoplasia related disorders in a subject, the method comprising administering to a subject a combination of a 1,2-diphenylpyrrole derivative and an aromatase inhibitor wherein the combination reduces the dosage compared to the dosage used in conventional treatment for neoplasia and/or neoplasia-related disorders.

In one embodiment, the invention provides a method of treating neoplasia and/or neoplasia related disorders, the method comprising administering a combination of a 1,2-diphenylpyrrole derivative and an aromatase inhibitor

In one embodiment, the invention provides a composition for treating cancer comprising, a combination of a 1,2-diphenylpyrrole derivative and an aromatase inhibitor or their respective pharmaceutically acceptable salts, solvates or prodrugs.

In one embodiment, the invention provides a method of inducing differentiation of tumor cells, the method comprising contacting the cells with an effective amount of a combination comprising a 1,2-diphenylpyrrole derivative and an estrogen receptor antagonist whereby the combination induces differentiation of tumor cells.

In one embodiment, the invention provides a method of inhibiting proliferation of cancer cells, the method comprising contacting a cancer cell with a combination comprising a 1,2-diphenylpyrrole derivative and an estrogen receptor antagonist whereby the combination inhibits proliferation of cancer cells.

In one embodiment, the invention provides a method for reducing proliferation of cancer cells, the method comprising delivering to the cells a combination comprising a 1,2-diphenylpyrrole derivative and an estrogen receptor antagonist, whereby the reduction of cell proliferation is greater than a reduction caused by either a 1,2-diphenylpyrrole derivative alone or an estrogen receptor antagonist alone.

In one embodiment, the invention provides a method of inhibiting metastases of tumor cells, the method comprising administering an effective amount of a combination comprising a 1,2-diphenylpyrrole derivative and an estrogen receptor antagonist such that the combination inhibits metastatic activity of tumor cells.

In one embodiment, the invention provides a method for inducing apoptosis in cancer cells, the method comprising contacting the cancer cells with a combination comprising a 1,2-diphenylpyrrole derivative and an estrogen receptor antagonist sufficient to induce apoptosis.

In one embodiment, the invention provides a method for sensitizing estrogen receptor antagonist resistant cancer cells to an estrogen receptor antagonist, the method comprising administering a combination comprising a 1,2-diphenylpyrrole derivative and an estrogen receptor antagonist wherein the combination sensitizes the cancer cells to the estrogen receptor antagonist.

In one embodiment, the invention provides a method of treating estrogen receptor antagonist resistance in a cancer cell, the method comprising administering a combination comprising a 1,2-diphenylpyrrole derivative and an estrogen receptor antagonist.

In one embodiment, the invention provides a method of modulating prostaglandin synthesis in a cancer cell, the method comprising contacting the cell with a combination comprising a 1,2-diphenylpyrrole derivative and an estrogen receptor antagonist wherein the combination inhibits prostaglandin synthesis in a cancer cell.

In one embodiment, the invention provides a method of modulating cyclooxygenase expression in a cancer cell, the method comprising delivering to the cell a combination comprising a 1,2-diphenylpyrrole derivative and an estrogen receptor antagonist wherein the combination inhibits cyclooxygenase expression in a cancer cell.

In one embodiment, the invention provides a method of modulating angiogenesis in a cancer cell, the method comprising contacting the cell with a combination comprising a 1,2-diphenylpyrrole derivative and an estrogen receptor antagonist wherein the combination inhibits angiogenesis in a cancer cell.

In one embodiment, the invention provides a method of reducing the dosage in conventional treatment for neoplasia and/or neoplasia related disorders in a subject, the method comprising administering to a subject a combination of a 1,2-diphenylpyrrole derivative and an estrogen receptor antagonist wherein the combination reduces the dosage in conventional treatment for neoplasia and/or neoplasia-related disorders.

In one embodiment, the invention provides a method of treating neoplasia and/or neoplasia related disorders, the method comprising administering a combination of a 1,2-diphenylpyrrole derivative and an estrogen receptor antagonist.

In one embodiment, the invention provides a composition for treating cancer comprising, a combination of a 1,2-diphenylpyrrole derivative and an estrogen receptor antagonist or their respective pharmaceutically acceptable salts, solvates or prodrugs.

In one embodiment, the invention provides a method of treatment comprising administering a composition comprising a combination of a 1,2-diphenylpyrrole derivative and an aromatase inhibitor wherein the 1,2-diphenylpyrrole derivative is 2-(4-ethoxyphenyl)-4-methyl-1-(4-sulfamoylphenyl)-pyrrole.

In another embodiment, the invention provides a method of treatment comprising administering a composition comprising a combination of a 1,2-diphenylpyrrole derivative and an aromatase inhibitor, wherein the aromatase inhibitor is letrozole.

In yet another embodiment, the invention provides a method of treatment comprising administering a composition comprising a combination of a 1,2-diphenylpyrrole derivative and an aromatase inhibitor wherein the 1,2-diphenylpyrrole derivative is 2-(4-ethoxyphenyl)-4-methyl-1-(4-sulfamoylphenyl)-pyrrole and the aromatase inhibitor is letrozole.

In one embodiment, the invention provides a method of treatment comprising administering a composition comprising a combination of a 1,2-diphenylpyrrole derivative and an aromatase inhibitor wherein the composition is a single dosage form.

In a further embodiment, the invention provides a method of treatment comprising administering a composition comprising a combination of a 1,2-diphenylpyrrole derivative and an aromatase inhibitor wherein the composition is a single dosage form and wherein the single dosage form enhances patient compliance and/or reduces pill burden.

In one embodiment, the invention provides a method for treating cancer, tumors, and tumor-related disorders, comprising administering a combination according to the invention by a mode of administration comprising oral, parenteral, buccal, intranasal, epidural, sublingual, pulmonary, local, rectal, or transdermal administration.

In a further embodiment, the invention provides a method for treating cancer, tumors, and tumor-related disorders, comprising administering a combination according to the invention by parenteral administration selected from intravenous, subcutaneous, intrathecal, and intramuscular administration.

In one embodiment, the invention provides a method comprising administering the combination in a single dosage form.

In yet a further embodiment, the invention provides a method of treatment comprising administering a composition comprising a combination of a 1,2-diphenylpyrrole derivative and an aromatase inhibitor in a single dosage form wherein the single dosage form is a single capsule or a single tablet.

In one embodiment, the invention provides a method of treatment comprising administering a composition comprising a combination of a 1,2-diphenylpyrrole derivative and an aromatase inhibitor wherein the composition is in the form of a single tablet.

In another embodiment, the invention provides a method of treatment comprising administering a composition comprising a combination of a 1,2-diphenylpyrrole derivative and an aromatase inhibitor wherein the single tablet comprises from about 100 mg to about 1200 mg of 2-(4-ethoxyphenyl)-4-methyl-1-(4-sulfamoylphenyl)-pyrrole and from about 1.0 mg to about 3.0 mg of letrozole.

In yet another embodiment, the invention provides a method of treatment comprising administering a composition comprising a combination of a 1,2-diphenylpyrrole derivative and an aromatase inhibitor wherein the single tablet comprises from about 100 mg to about 1200 mg of 2-(4-ethoxyphenyl)-4-methyl-1-(4-sulfamoylphenyl)-pyrrole and about 1.0 mg of letrozole.

In one embodiment, the invention provides a method of treatment comprising administering a single tablet composition comprising a combination of a 1,2-diphenylpyrrole derivative and an aromatase inhibitor wherein the single tablet comprises from about 100 mg to about 1200 mg of 2-(4-ethoxyphenyl)-4-methyl-1-(4-sulfamoylphenyl)-pyrrole and about 1.5 mg of letrozole.

In another embodiment, the invention provides a method of treatment comprising administering a composition comprising a combination of a 1,2-diphenylpyrrole derivative and an aromatase inhibitor wherein the single tablet comprises from about 100 mg to about 1200 mg of 2-(4-ethoxyphenyl)-4-methyl-1-(4-sulfamoylphenyl)-pyrrole and about 2.0 mg of letrozole.

In another embodiment, the invention provides a method of treatment comprising administering a composition comprising a combination of a 1,2-diphenylpyrrole derivative and an aromatase inhibitor wherein the single tablet comprises from about 100 mg to about 1200 mg of 2-(4-ethoxyphenyl)-4-methyl-1-(4-sulfamoylphenyl)-pyrrole and about 2.5 mg of letrozole.

In another embodiment, the invention provides a method of treatment comprising administering a composition comprising a combination of a 1,2-diphenylpyrrole derivative and an aromatase inhibitor wherein the single tablet comprises from about 100 mg to about 1200 mg of 2-(4-ethoxyphenyl)-4-methyl-1-(4-sulfamoylphenyl)-pyrrole and from about 0.5 mg to about 1.5 mg of anastrozole.

In another embodiment, the invention provides a method of treatment comprising administering a composition comprising a combination of a 1,2-diphenylpyrrole derivative and an aromatase inhibitor wherein the single tablet comprises from about 100 mg to about 1200 mg of 2-(4-ethoxyphenyl)-4-methyl-1-(4-sulfamoylphenyl)-pyrrole and about 0.5 mg of anastrozole.

In another embodiment, the invention provides a method of treatment comprising administering a composition comprising a combination of a 1,2-diphenylpyrrole derivative and an aromatase inhibitor wherein the single tablet comprises from about 100 mg to about 1200 mg of 2-(4-ethoxyphenyl)-4-methyl-1-(4-sulfamoylphenyl)-pyrrole and about 1.0 mg of anastrozole.

In another embodiment, the invention provides a method of treatment comprising administering a composition comprising a combination of a 1,2-diphenylpyrrole derivative and an aromatase inhibitor wherein the single tablet comprises from about 100 mg to about 1200 mg of 2-(4-ethoxyphenyl)-4-methyl-1-(4-sulfamoylphenyl)-pyrrole and about 1.5 mg of anastrozole.

In another embodiment, the invention provides a method of treatment comprising administering a composition comprising a combination of a 1,2-diphenylpyrrole derivative and an aromatase inhibitor wherein the single tablet comprises from about 100 mg to about 1200 mg of 2-(4-ethoxyphenyl)-4-methyl-1-(4-sulfamoylphenyl)-pyrrole and from about 5 mg to about 35 mg of exemestane.

In another embodiment, the invention provides a method of treatment comprising administering a composition comprising a combination of a 1,2-diphenylpyrrole derivative and an aromatase inhibitor wherein the single tablet comprises from about 100 mg to about 1200 mg of 2-(4-ethoxyphenyl)-4-methyl-1-(4-sulfamoylphenyl)-pyrrole and about 5 mg of exemestane.

In another embodiment, the invention provides a method of treatment comprising administering a composition comprising a combination of a 1,2-diphenylpyrrole derivative and an aromatase inhibitor wherein the single tablet comprises from about 100 mg to about 1200 mg of 2-(4-ethoxyphenyl)-4-methyl-1-(4-sulfamoylphenyl)-pyrrole and about 12 mg of exemestane.

In another embodiment, the invention provides a method of treatment comprising administering a composition comprising a combination of a 1,2-diphenylpyrrole derivative and an aromatase inhibitor wherein the single tablet comprises from about 100 mg to about 1200 mg of 2-(4-ethoxyphenyl)-4-methyl-1-(4-sulfamoylphenyl)-pyrrole and about 25 mg of exemestane.

In another embodiment, the invention provides a method of treatment comprising administering a composition comprising a combination of a 1,2-diphenylpyrrole derivative and an aromatase inhibitor wherein the single tablet comprises from about 100 mg to about 1200 mg of 2-(4-ethoxyphenyl)-4-methyl-1-(4-sulfamoylphenyl)-pyrrole and about 35 mg of exemestane.

In one embodiment, the invention provides a method of treatment comprising administering a composition comprising a combination of a 1,2-diphenylpyrrole derivative and an aromatase inhibitor wherein the composition is suitable for once-daily administration.

In another embodiment, the invention provides a method of treatment comprising administering a composition comprising a combination of a 1,2-diphenylpyrrole derivative and an aromatase inhibitor wherein the aromatase inhibitor is a small molecule compound.

In a further embodiment, the invention provides a method of treatment comprising administering a composition comprising a combination of a 1,2-diphenylpyrrole derivative and a small molecule wherein the small molecule compound is selected from the group consisting of letrozole, anastrozole and exemestane.

In yet another embodiment, the invention provides a method of treatment comprising administering a composition comprising a combination of a 1,2-diphenylpyrrole derivative and an aromatase inhibitor wherein the composition contains a lower dose than a conventional treatment for cancer.

In a further embodiment, the invention provides a method of treatment comprising administering a composition comprising a combination of a 1,2-diphenylpyrrole derivative and an aromatase inhibitor wherein the composition reduces the side effects of cancer treatment.

In yet a further embodiment, the invention provides a method of treatment comprising administering a composition comprising a combination of a 1,2-diphenylpyrrole derivative and an aromatase inhibitor wherein the composition enhances treatment of cancer.

### Pharmaceutical Compositions

In one embodiment the invention provides a pharmaceutical composition for treating cancer comprising, a combination of a 1,2-diphenylpyrrole derivative and an aromatase inhibitor, and a pharmaceutically acceptable excipient or carrier.

In one embodiment, the invention provides a pharmaceutical composition for treating cancer wherein the 1,2-diphenylpyrrole derivative is 2-(4-ethoxyphenyl)-4-methyl-1-(4-sulfamoylphenyl)-pyrrole.

In another embodiment, the invention provides a pharmaceutical composition for treating cancer wherein the aromatase inhibitor is letrozole.

In yet another embodiment, the invention provides a pharmaceutical composition for treating cancer wherein the 1,2-diphenylpyrrole derivative is 2-(4-ethoxyphenyl)-4-methyl-1-(4-sulfamoylphenyl)-pyrrole and the aromatase inhibitor is letrozole.

In another embodiment, the invention provides a pharmaceutical composition for treating cancer wherein the aromatase inhibitor is anastrozole.

In yet another embodiment, the invention provides a pharmaceutical composition for treating cancer wherein the 1,2-diphenylpyrrole derivative is 2-(4-ethoxyphenyl)-4-methyl-1-(4-sulfamoylphenyl)-pyrrole and the aromatase inhibitor is anastrozole.

In another embodiment, the invention provides a pharmaceutical composition for treating cancer wherein the aromatase inhibitor is exemestane.

In yet another embodiment, the invention provides a pharmaceutical composition for treating cancer wherein the 1,2-diphenylpyrrole derivative is 2-(4-ethoxyphenyl)-4-methyl-1-(4-sulfamoylphenyl)-pyrrole and the aromatase inhibitor is exemestane.

In one embodiment the invention provides a pharmaceutical composition for treating cancer comprising, a combination of a 1,2-diphenylpyrrole derivative and an estrogen receptor antagonist, and a pharmaceutically acceptable excipient or carrier.

In one embodiment, the invention provides a pharmaceutical composition for treating cancer wherein the 1,2-diphenylpyrrole derivative is 2-(4-ethoxyphenyl)-4-methyl-1-(4-sulfamoylphenyl)-pyrrole.

In another embodiment, the invention provides a pharmaceutical composition for treating cancer wherein the estrogen receptor antagonist is tamoxifen.

In yet another embodiment, the invention provides a pharmaceutical composition for treating cancer wherein the 1,2-diphenylpyrrole derivative is 2-(4-ethoxyphenyl)-4-methyl-1-(4-sulfamoylphenyl)-pyrrole and the estrogen receptor antagonist is tamoxifen.

In another embodiment, the invention provides a pharmaceutical composition for treating cancer wherein the estrogen receptor antagonist is raloxifene.

In yet another embodiment, the invention provides a pharmaceutical composition for treating cancer wherein the 1,2-diphenylpyrrole derivative is 2-(4-ethoxyphenyl)-4-methyl-1-(4-sulfamoylphenyl)-pyrrole and the estrogen receptor antagonist is raloxifene.

In another embodiment, the invention provides a pharmaceutical composition for treating cancer wherein the estrogen receptor antagonist is toremifene.

In yet another embodiment, the invention provides a pharmaceutical composition for treating cancer wherein the 1,2-diphenylpyrrole derivative is 2-(4-ethoxyphenyl)-4-methyl-1-(4-sulfamoylphenyl)-pyrrole and the estrogen receptor antagonist is toremifene.

In another embodiment, the invention provides a pharmaceutical composition for treating cancer wherein the estrogen receptor antagonist is fulvestrant.

In yet another embodiment, the invention provides a pharmaceutical composition for treating cancer wherein the 1,2-diphenylpyrrole derivative is 2-(4-ethoxyphenyl)-4-methyl-1-(4-sulfamoylphenyl)-pyrrole and the estrogen receptor antagonist is fulvestrant.

### Kits/Articles of Manufacture

In one embodiment the invention provides a kit for treating cancer comprising a single dosage form comprising a combination of a 1,2-diphenylpyrrole derivative and an aromatase inhibitor and instructions on administration.

In one embodiment the invention provides a kit for treating cancer having a composition comprising a combination of a 1,2-diphenylpyrrole derivative and an aromatase inhibitor wherein the 1,2-diphenylpyrrole derivative is 2-(4-ethoxyphenyl)-4-methyl-1-(4-sulfamoylphenyl)-pyrrole and the aromatase inhibitor is selected from letrozole, anastrozole or exemestane.

In one embodiment the invention provides a kit for treating cancer comprising a single dosage form comprising a combination of a 1,2-diphenylpyrrole derivative and an estrogen receptor antagonist and instructions on administration.

In one embodiment the invention provides a kit for treating cancer having a composition comprising a combination of a 1,2-diphenylpyrrole derivative and an aromatase inhibitor wherein the 1,2-diphenylpyrrole derivative is 2-(4-ethoxyphenyl)-4-methyl-1-(4-sulfamoylphenyl)-pyrrole and the estrogen receptor antagonist is selected from tamoxifen, toremifene, raloxifene or fulvestrant.

### INCORPORATION BY REFERENCE

All publications, patents, and patent applications described in this specification are herein incorporated by reference to the same extent as if each individual publication, patent, or patent application was specifically and individually indicated to be incorporated by reference.

### BRIEF DESCRIPTION OF THE DRAWINGS

FIG. 1 provides graphs illustrating COX-2 expression levels in colorectal cancer. The overall 10-year survival curves of patients with Cox-2 negative and Cox-2 positive are shown for the entire cohort, P=0.0006 (A), as well as for patients with stage I/II, P=0.0271 (B), or stage III, P=0.0081 (C) disease.

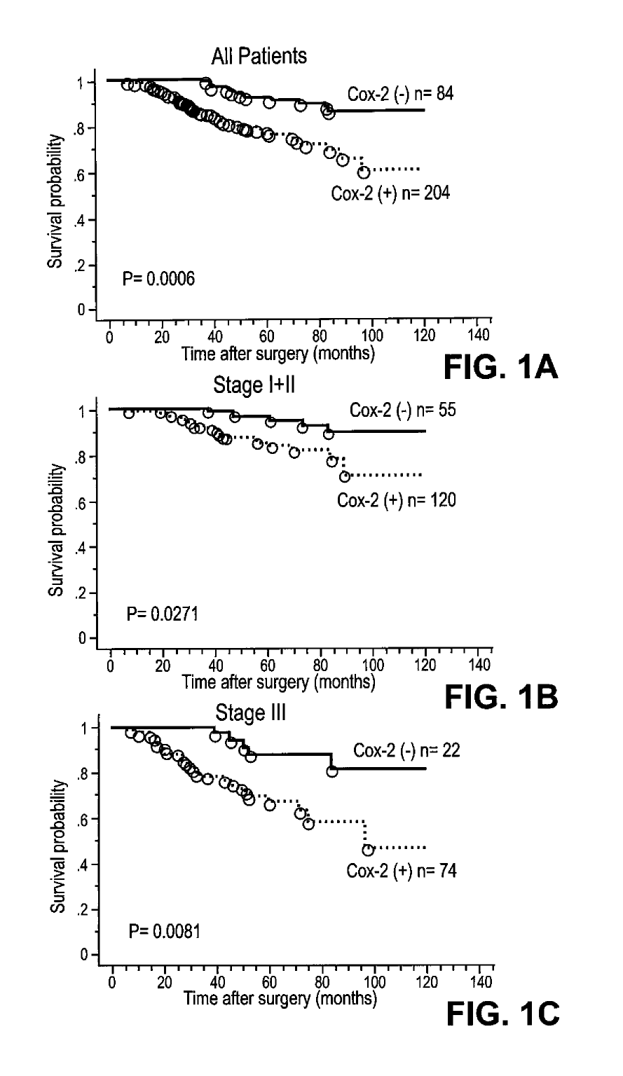

### DETAILED DESCRIPTION

Provided herein are methods of treating cancer based on the administration of a combination of a 1,2-diphenylpyrrole derivative and an aromatase inhibitor. The methods may further include treatments wherein the combination is supplemented with one or more therapeutic agents or therapies. In one method, lapatinib, alone or in further combination with capecitabine is administered in combination with the 1,2-diphenylpyrrole and the aromatase inhibitor. The latter combination is useful in the treatment of breast cancers associated with overexpression of Her-2/neu. The 1,2-diphenylpyrrole derivative and the aromatase inhibitor may be provided in separate dosage forms or combined in one dosage form (e.g. a fixed dose).

Also provided herein are methods of treating cancer based on the administration of a combination of a 1,2-diphenylpyrrole derivative (a COX-2 selective inhibitor) and an estrogen receptor antagonist. The methods may further include treatments wherein the combination is supplemented with one or more therapeutic agents or therapies. In one method, lapatinib, alone or in further combination with capecitabine is administered in combination with the 1,2-diphenylpyrrole and an estrogen receptor antagonist. The latter combination is useful in the treatment of breast cancers associated with overexpression of Her-2/neu. The 1,2-diphenylpyrrole derivative and the estrogen receptor antagonist may be provided in separate dosage forms or combined in one dosage form (e.g. a fixed dose)

To facilitate understanding of the disclosure set forth herein, a number of terms are defined below.

As used herein, “abnormal cell growth,” refers to cell growth that is independent of normal regulatory mechanisms (e.g., loss of contact inhibition), including the abnormal growth of normal cells and the growth of abnormal cells.

“Neoplasia” as described herein, is an abnormal, unregulated and disorganized proliferation of cells that is distinguished from normal cells by autonomous growth and somatic mutations. As neoplastic cells grow and divide they pass on their genetic mutations and proliferative characteristics to progeny cells. A neoplasm, or tumor, is an accumulation of neoplastic cells. In some embodiments, the neoplasm can be benign or malignant.

“Metastasis,” as used herein, refers to the dissemination of tumor cells via lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

As discussed herein, “angiogenesis” is prominent in tumor formation and metastasis. Angiogenic factors have been found associated with several solid tumors such as rhabdomyosarcomas, retinoblastoma, Ewing sarcoma, neuroblastoma, and osteosarcoma. A tumor cannot expand without a blood supply to provide nutrients and remove cellular wastes. Tumors in which angiogenesis is important include solid tumors such as renal cell carcinoma, hepatocellular carcinoma, and benign tumors such as acoustic neuroma, and neurofibroma. Angiogenesis has been associated with blood-born tumors such as leukemias. It is believed that angiogenesis plays a role in the abnormalities in the bone marrow that give rise to leukemia. Prevention of angiogenesis could halt the growth of cancerous tumors and the resultant damage to the subject due to the presence of the tumor.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” are meant to include alleviating or abrogating a disorder, disease, or condition; or one or more of the symptoms associated with the disorder, disease, or condition; or alleviating or eradicating the cause(s) of the disorder, disease, or condition itself.

The term “therapeutically effective amount” refers to the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

### Cyclooxygenase

Cyclooxygenase (COX) is an enzyme that is responsible for the formation of important biological mediators called prostanoids, including prostaglandins, prostacyclin and thromboxane. COX converts arachidonic acid, an ω-6 essential fatty acid, to prostaglandin H₂ (PGH₂), the precursor of the series-2 prostanoids. The enzyme contains two active sites: a heme with peroxidase activity, responsible for the reduction of PGG₂ to PGH₂, and a cyclooxygenase site, where arachidonic acid is converted into the hydroperoxy endoperoxide prostaglandin G₂ (PGG₂). The reaction proceeds through a hydrogen atom abstraction from arachidonic acid by a tyrosine radical generated by the peroxidase active site, then two oxygen molecules react with the arachidonic acid radical, giving PGG₂.

COX-1 is a constitutive enzyme responsible for biosynthesis of prostaglandins in the gastric mucosa and in the kidney among other sites. COX-2 is an enzyme that is produced by an inducible gene that is responsible for biosynthesis of prostaglandins in inflammatory cells. Inflammation causes induction of COX-2, leading to release of prostanoids (prostaglandin E2), which sensitize peripheral nociceptor terminals and produce localized pain hypersensitivity, inflammation and edema.

### Overexpression of COX-2 and Cancer

The overexpression of COX-2 and also the upstream and downstream enzymes of the prostaglandin synthesis pathway has been demonstrated in multiple cancer types and some pre-neoplastic lesions. Direct interactions of prostaglandins with their receptors through autocrine or paracrine pathways to enhance cellular survival or stimulate angiogenesis have been proposed as molecular mechanisms underlying the pro-carcinogenic functions of COX enzymes.

Studies indicate that prostaglandins synthesized by cyclooxygenase play a role in the initiation and promotion of cancer. Aberrant COX-2 expression was reported in colorectal carcinomas and adenomas, and has been detected in various human cancers, including those of the breast. Moreover, COX-2 is overexpressed in neoplastic lesions of the colon, breast, lung, prostate, esophagus, pancreas, intestine, cervix, ovaries, urinary bladder and head and neck (see Table 1 below).

TABLE 1
COX-2 Expression in Tumors

| Tumor Type | % Tissue expressing COX-2 |
|---|---|
| Colorectal Cancer | 70-95 |
| Non-small Cell Lung Cancer | 70-90 |
| Gastric Cancer | 45-75 |
| Pancreatic Cancer | 40-80 |
| Glioblastoma Multiforme | 40-70 |
| Bladder Cancer | 50-60 |
| Esophageal Cancer | 50-60 |
| Breast Cancer | 40-50 |
| Ovarian Cancer | 40-60 |
| Prostate Cancer | 40-60 |

COX-2overexpression in murine mammary glands is sufficient to cause tumor formation. In several in vitro and animal models, COX-2 inhibitors have inhibited tumor growth and metastasis.

In addition to cancers per se, COX-2 is also expressed in the angiogenic vasculature within and adjacent to hyperplastic and neoplastic lesions indicating that COX-2 plays a role in angiogenesis. In both the mouse and rat, COX-2 inhibitors markedly inhibited bFGF-induced neovascularization. The utility of COX-2 inhibitors as chemopreventive, antiangiogenic and chemotherapeutic agents is described in the literature (Koki et al., Exp. Opin., Invest. Drugs, 1999, 8(10) 1623-38).

### Aromatase

Estrogen is one of the female sex hormones and has many functions in the body. It has been found that about 80% of breast cancer tumors overexpress the estrogen receptor and respond positively to the presence of estrogen. In postmenopausal women, ovarian estrogen production is reduced and plasma estrogen levels are generally lower than in premenopausal women.

A residual source of estrogen in post-menopausal women is the synthesis of estrogens from androgens, which is catalyzed by aromatase. Inhibition of aromatase activity should lead to a reduction in the levels of estrogen and therefore a reduction in the growth of breast cancer tumors which respond positively to the presence of estrogen.

Aromatase is an enzyme of the cytochrome P450 family and a product of the CYP19 gene. The chemical function of aromatase is to convert testosterone to estradiol and androstenedione to estrone.

### Aromatase and COX Pathways

Studies of the relationship between the aromatase and COX pathways have been undertaken (Subbaramaiah et al Cancer Research 2006, 66(10), 5504-11). This investigation into the regulation of CYP19 gene expression has shown that COX-2-derived PGE₂ acts to stimulate CYP19 gene expression and therefore aromatase activity. This correlates with the observation that COX-2 and aromatase are both highly expressed in many human breast cancer specimens. These investigators also reported that mammary aromatase activity is regulated by COX-2 and additionally found that genetic or pharmacological inhibition of COX-2 results in aromatase activity reduction. A model has been proposed wherein HER2/neu stimulates aromatase activity via COX-2 upregulation and consequent increase in PGE₂ production.

### Breast Cancer

Today, among women in the United States, breast cancer remains the most frequent diagnosed cancer. One in 8 women in the United States is at risk of developing breast cancer. Age, family history, diet, and genetic factors have been identified as risk factors for breast cancer. Breast cancer is the second leading cause of death among women.

### HER2/neu Positive Breast Cancer

Cancers associated with overexpression of HER2/neu include breast, ovarian, endometrial, prostate, gastric, salivary gland, pancreatic, colorectal, oral and non-small cell lung cancers. Breast cancer has been a focus of anti-HER2/neu treatments.

Approximately 25-30 percent of breast cancers have an amplification of the HER2/neu gene or overexpression of its protein product. Overexpression of this receptor in breast cancer is associated with increased disease recurrence and worse prognosis.

In the treatment of HER2/neu positive breast cancer, the therapies and compositions described herein may be combined with other antiangiogenic agents, or in combination with surgery, radiation therapy or with chemotherapeutic agents, including, for example, lapatinib, alone or in further combination with capecitabine, trastuzumab, CL-387785, paclitaxel, docetaxel, cisplatin, or carboplatin

### Hormone Positive Cancer

Many breast cancers require the hormone estrogen to grow. In women who have had their menopause, the main source of estrogen is through the conversion of androgens into estrogens. As discussed above, this process is carried out by the aromatase enzyme. In the treatment of hormone positive breast cancer, the therapies and compositions described herein may be advantageously combined with one or more cancer treatments.

### Triple Negative Breast Cancer

In the treatment of triple negative breast cancer wherein the cancer is estrogen receptor-negative, progesterone receptor-negative and HER2-negative, compositions and therapies described herein may be combined with other therapeutic agents. Such agents include, by way of example only, cetuximab, paclitaxel, docetaxel, taxane formulations, for example, Abraxane® (ABI-007), Paclitaxel-Cremophor EL, Paclitaxel poliglumex, and Paclitaxel injectable emulsion (PIE). These combinations may be advantageous when the cancer association with HER2 overexpression is present but undetected due to technical limitations in tests employed in quantifying HER 2 expression.

As discussed above, provided herein are cancer treatments based on the combination of the compound 2-(4-ethoxyphenyl)-4-methyl-1-(4-sulfamoylphenyl)-pyrrole and an aromatase inhibitor. Also provided herein are cancer treatments based on the combination of the compound 2-(4-ethoxyphenyl)-4-methyl-1-(4-sulfamoylphenyl)-pyrrole and an antiestrogen. Combinations based on 2-(4-ethoxyphenyl)-4-methyl-1-(4-sulfamoylphenyl)-pyrrole have shown synergistic advantages superior to the effects obtained with other COX-2 inhibitors, such as celecoxib.

The compounds that form the base combinations provided herein are described in more detail below.

### 2-(4-Ethoxyphenyl)-4-methyl-1-(4-sulfamoylphenyl)-pyrrole

2-(4-Ethoxyphenyl)-4-methyl-1-(4-sulfamoylphenyl)-pyrrole is a COX-2 selective inhibitor. U.S. Pat. No. 6,887,893 and RE39,420 describe the preparation of 2-(4-ethoxyphenyl)-4-methyl-1-(4-sulfamoylphenyl)-pyrrole and other chemically-related compounds.

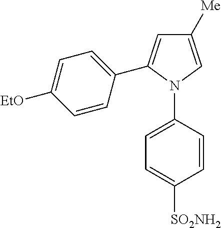

### Chemical structure of 2-(4-ethoxyphenyl)-4-methyl-1-(4-sulfamoylphenyl)-pyrrole

### Aromatase Inhibitors

Aromatase inhibitors decrease the body's estrogen by blocking the enzyme aromatase from turning androgen into estrogen. For the treatment of early stage breast cancer, certain aromatase inhibitors may be used as adjuvant therapy instead of tamoxifen or after 2 or more years of tamoxifen. For the treatment of metastatic breast cancer, aromatase inhibitors are being tested in clinical trials to compare them to hormone therapy with tamoxifen.

As described herein, an “aromatase inhibitor” is a molecule which inhibits the activity of the aromatase enzyme. Compounds which are inhibitors of aromatase can be readily identified by one skilled in the art using methods such as, for example, standard pharmacological test procedures which measure the inhibition of the conversion of 1,2-³H-androstenedione to estrone.

In brief, a microsomal fraction is prepared from human placenta by the method as described by Thompson and Siiteri (J. Biol. Chem., Vol. 249, p. 5364 (1974)). The microsomal preparation so obtained is lyophilized and stored at −40 ° C. The human placental microsomes are added to 1,2-³H-androstenedione and incubated for 20 minutes at 37° C. The amount of aromatization of the labelled substrate is detected by the loss of ³H₂O into the incubation medium. The substrate is removed by chloroform extraction, followed by adsorption to charcoal in suspension. The charcoal is removed by centrifugation and the steroid-free medium is counted in a liquid scintillation counter. Compositions are tested for aromatase inhibitory activity by adding them to the incubation medium prior to the addition of the microsomes. The relative cpm obtained with and without the composition is used to calculate the percent inhibition of the aromatization of androstenedione to estrone. IC₅₀ values can be determined graphically as the concentration of test composition at which the aromatization of androstenedione to estrone is reduced to 50% of control value.

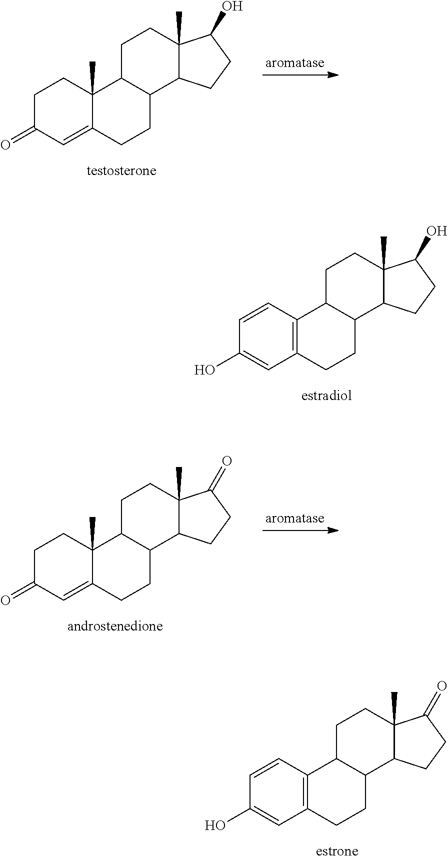

Subcutaneous fat is a major site of aromatase activity and it has been suggested that plasma estrogen levels correlate with body-mass index (Longcope et al , Metabolism 1986, 35, 235-7). It has been suggested that at menopause, plasma estrogen levels fall from about 110 pg/mL to a much lower level of about 7 pg/mL. However, in post-menopausal women, the intra-tumoral concentration of estradiol has been found to be about 10 times higher than in the plasma, probably due to aromatase activity within the tumor.

Inhibition of aromatase as a treatment option for breast cancer has been studied with some success. Currently three aromatase inhibitors are approved for marketing in the US for the treatment of breast cancer, at various stages, in post-menopausal women. Letrozole (Femara®) is indicated for several treatment options including, extended adjuvant treatment of early breast cancer in postmenopausal women with 5 years prior tamoxifen treatment, treatment of post menopausal women with hormone receptor positive (or unknown) locally advanced or metastatic breast cancer and advanced breast cancer treatment in postmenopausal women with disease progression following antiestrogen therapy.

Anastrozole (Arimidex®) is indicated for several treatment options including, adjuvant treatment of postmenopausal women with hormone receptor-(+) early breast cancer, first-line treatment of post menopausal women with hormone receptor-(+) (or unknown) locally advanced or metastatic breast cancer and advanced breast cancer in postmenopausal women with disease progression following tamoxifen therapy.

Exemestane (Aromasin®) is indicated for several treatment options including, adjuvant treatment of postmenopausal women with estrogen-receptor-(+) early breast cancer who have received 2-3 years of tamoxifen treatment and advanced breast cancer in postmenopausal women with disease progression following tamoxifen therapy.

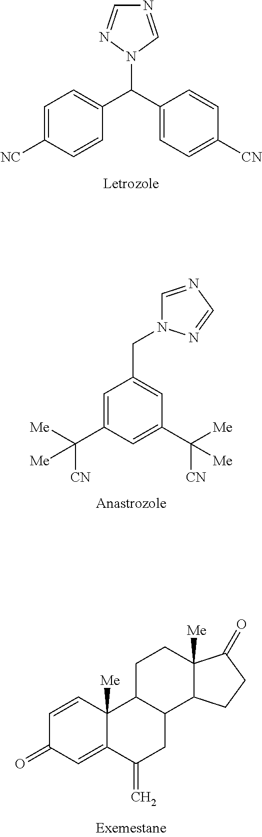

These drugs are grouped into two classes: (Type 1) exemestane is based on a steroid chemical structure and (type 2) letrozole and anastrozole are based on a non-steroidal chemical structure. Clinical trials have shown letrozole to be superior to tamoxifen in the treatment of advanced ER(+) disease. In early disease, adjuvant therapy with anastrozole appears to be superior to therapy with tamoxifen in reducing risk of relapse. Recent clinical trial results have led to aromatase inhibitors replacing tamoxifen as the standard of care for breast cancer treatment.

Provided herein is a method for treating a subject having cancer, comprising administering to the subject, a therapeutically effective amount of a combination comprising a 1,2-diphenylpyrrole derivative and an aromatase inhibitor or their respective pharmaceutically acceptable salts, solvates, polymorphs or prodrugs wherein the 1,2-diphenylpyrrole derivative is 2-(4-ethoxyphenyl)-4-methyl-1-(4-sulfamoylphenyl)-pyrrole and the aromatase inhibitor is selected from letrozole, anastrozole or exemestane.

### Estrogen Receptor Antagonists (Antiestrogens)

Besides reducing the levels of endogenous estrogen by inhibiting its synthesis, another approach to countering the tumor promoting effect of estrogen is the use of compounds that serve as estrogen antagonists at the estrogen receptor. Four such compounds are approved for this use: fulvestrant, tormifene, tamoxifen and raloxifene,

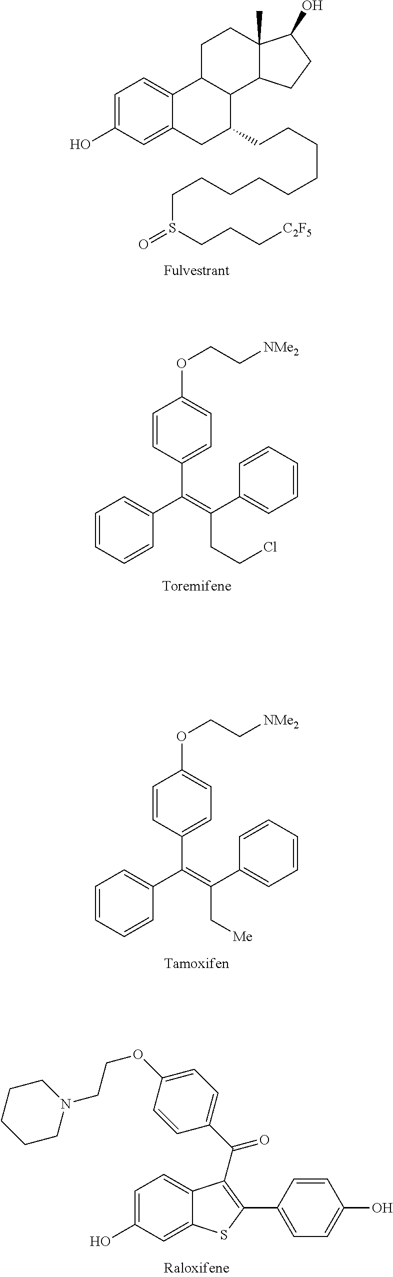

Fulvestrant (Fasiodex®) is an estrogen receptor antagonist that binds the estrogen receptor with an affinity comparable to estradiol. This binding event results in a downregulation of ER gene expression in human breast cancer cells. Fulvestrant is indicated for the treatment of hormone receptor positive metastatic breast cancer in post menopausal women with disease progression following antiestrogen therapy. The recommended dose of fulvestrant is 250 mg administered by a single injection intramuscularly once a month.

Toremifene (Fareston®) is a non-steroidal predominantly anti-estrogenic compound of the triphenylethylene class. The antitumor effects of toremifene are thought to be due to its anti-estrogenic activity wherein it is able to compete with estrogen for the estrogen receptor. Toremifene is indicated for the treatment of metastatic breast cancer in post menopausal women with hormone receptor positive or unknown tumors. The dosage of toremifene is 60 mg taken orally once per day.

Tamoxifen is a non-steroidal predominantly anti-estrogenic compound of the triphenylethylene class. The antitumor effects of tamoxifen are thought to be due to its anti-estrogenic activity wherein it is able to compete with estrogen for the estrogen receptor. Tamoxifen is indicated in the treatment of metastatic breast cancer in women and men. In premenopausal women with metastatic breast cancer tamoxifen is an alternative to oophorectomy or ovarian irradiation.

A positive correlation between ER status and response to tamoxifen treatment has been suggested. In the adjuvant setting, tamoxifen is indicated following surgery or radiation therapy. The dosage of tamoxifen is 10-20 mg taken orally twice per day.

Raloxifene is an estrogen agonist/antagonist of the benzothiophene class, commonly referred to as a selective estrogen receptor modulator (SERM). The biological actions of raloxifene are largely mediated through binding to estrogen receptors. This binding results in activation of estrogenic pathways in some tissues (agonism) and blockade of estrogenic pathways in others (antagonism). Raloxifene appears to act as an estrogen agonist in bone. It decreases bone resorption and bone turnover, increases bone mineral density and decreases fracture incidence. Other data demonstrate that raloxifene is an estrogen antagonist in uterine and breast tissues. Raloxifene is indicated for reduction in risk of invasive breast cancer in postmenopausal women with osteoporosis or at high risk of invasive breast cancer.

Provided herein is a method for treating a subject having cancer, comprising administering to the subject, a therapeutically effective amount of a combination comprising a 1,2-diphenylpyrrole derivative and an estrogen receptor antagonist or their respective pharmaceutically acceptable salts, solvates, polymorphs or prodrugs wherein the 1,2-diphenylpyrrole derivative is 2-(4-ethoxyphenyl)-4-methyl-1-(4-sulfamoylphenyl)-pyrrole and the estrogen receptor antagonist is selected from fulvestrant, tormifene, raloxifene or tamoxifen.

### Additional Therapy

Available additional treatments for breast cancer that may be advantageously employed in combination with the therapies and compositions disclosed herein include, without limitation, radiation therapy, chemotherapy, antibody therapy, and tyrosine kinase inhibitors as adjuvant therapy.

Radiation therapy is a cancer treatment that uses high-energy x-rays or other types of radiation to kill cancer cells or keep them from growing. Chemotherapy is a cancer treatment that uses drugs to stop the growth of cancer cells, either by killing the cells or by stopping them from dividing. When chemotherapy is taken by mouth or injected into a vein or muscle, the drugs enter the bloodstream and can reach cancer cells throughout the body (systemic chemotherapy). When chemotherapy is placed directly into the spinal column, an organ, or a body cavity such as the abdomen, the drugs mainly affect cancer cells in those areas (regional chemotherapy). The way the chemotherapy is given depends on the type and stage of the cancer being treated.

Different chemotherapeutic agents are known in the art for treating breast cancer. Cytoxic agents used for treating breast cancer include doxorubicin, cyclophosphamide, methotrexate, 5-fluorouracil, mitomycin C, mitoxantrone, paclitaxel, taxane formulations such as by way of example only, Abraxane® (ABI-007), Paclitaxel-Cremophor EL, Paclitaxel poliglumex, and Paclitaxel injectable emulsion (PIE), gemcitabine, docetaxel, capecitabine and epirubicin.

Other chemotherapy against breast cancer includes treatment with one or more of bendamustine, carboplatin (for example, Paraplatin®), carmustine (for example, BCNU®), chlorambucil (for example, Leukeran®), cisplatin (for example, Platinol®), cyclophosphamide injection (for example, Cytoxan®), oral cyclophosphamide (for example, Cytoxan®), dacarbazine (for example, DTIC®), ifosfamide (for example, ifex®), lomustine (for example, CCNU®), mechlorethamine (for example, nitrogen mustard, Mustargen®), melphalan (for example, Alkeran®), procarbazine (for example, Matulane®), bleomycin (for example, Blenoxane®), doxorubicin (for example, Adriamycin®, Rubex®), epirubicin, Idarubicin (for example, Idamycin®), mitoxantrone (for example, Novantrone®), gemcitabine (for example, Gemzar®), oral mercaptopurine (for example, Purinethol®). methotrexate, pentostatin IV (for example, Nipent®), oral thioguanine (for example, Lanvis®), oral etoposide (for example, VP-16, VePesid®, Etopophos) etoposide IV (for example, VP-16, VePesid®, Etopophos), vinblastine (for example, Velban®), vincristine (for example, Oncovin®), vinorelbine (for example, Navelbine®), dexamethasone (for example, Decadron®), methylprednisolone (for example, Medrol®), and prednisone (for example, Deltasone®).

Monoclonal antibody therapy is a cancer treatment that uses antibodies made in the laboratory, from a single type of immune system cell. These antibodies can identify substances on cancer cells or normal substances that may help cancer cells grow. The antibodies attach to the substances and kill the cancer cells, block their growth, or keep them from spreading. Monoclonal antibodies are given by infusion. They may be used alone or to carry drugs, toxins, or radioactive material directly to cancer cells. Monoclonal antibodies are also used in combination with chemotherapy as adjuvant therapy.

Trastuzumab (Herceptin®) is a monoclonal antibody that blocks the effects of the growth factor protein HER2, which transmits growth signals to breast cancer cells.

Trastuzumab leads to clinical responses as a single agent and improves survival when added to chemotherapy for advanced HER2-positive breast cancer. However, some patients do not respond to trastuzumab, and most eventually develop clinical resistance. Mechanisms of intrinsic and acquired trastuzumab resistance are poorly understood. One study which utilized a cell line-based approach to delineate genetic and protein alterations associated with resistance has been reported (D. Tripathy et al Journal of Clinical Oncology, 2005 Vol 23, No 16S, 3121). These researchers studied two HER2-positive breast cancer cell lines (BT474 and SKBR3) that were serially passaged in the presence of trastuzumab until in vitro resistance was documented. Resistant cell lines emerged after 12 months and exhibited a 3-fold more rapid growth rate in the absence of trastuzumab. Following trastuzumab exposure, G₀/G₁ arrest was observed in sensitive compared to resistant cells (84 vs. 68%), with fewer cells in S-phase (3 vs. 14%). Resistant cell lines exhibited fewer changes in gene expression with trastuzumab as well as upregulation of the chemokine receptor CXCR4 and mitotic checkpoint regulators, and downregulation of PTEN compared to sensitive cells.

Additional, illustrative, treatments that may be advantageously combined with the compositions and therapies disclosed herein may include, without limitation, administration of agents including, but not limited to lapatinib, alone or in combination with capecitabine, docetaxel, epirubicin, epothilone A, B or D, goserelin acetate, paclitaxel, pamidronate, bevacizumab, or trastuzumab.

The compositions provided herein may be enantiomerically pure, such as a single enantiomer or a single diastereomer, or be stereoisomeric mixtures, such as a mixture of enantiomers, a racemic mixture, or a diastereomeric mixture, or a polymorph of the active agent. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include chiral synthesis from a suitable optically pure precursor or resolution of the racemate using, for example, chiral chromatography, recrystallization, resolution, diastereomeric salt formation, or derivatization into diastereomeric adducts followed by separation.

When the composition described herein contains an acidic or basic moiety, it may also be provided as a pharmaceutically acceptable salt (See, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonie acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The composition described herein may also be provided as a prodrug, which is a functional derivative of the 1,2-diphenylpyrrole derivative and/or the aromatase inhibitor and is readily convertible into the parent compound in vivo. Alternatively, the composition described herein may also be provided as a prodrug of the 1,2-diphenylpynole derivative and/or the estrogen receptor antagonist and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

### Oral Formulations

Oral formulations containing the active combinations described herein may comprise any conventionally used oral forms, including: tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups, buccal forms, and oral liquids. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. In some embodiments are surface modifying agents which include nonionic and anionic surface modifying agents. For example, surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

### Oral Administration

As described herein, the combination regimen can be given simultaneously or can be given in a staggered regimen, with a 1,2-diphenylpyrrole derivative being given at a different time during the course of chemotherapy than the aromatase inhibitor or the estrogen receptor antagonist. This time differential may range from several minutes, hours, days, weeks, or longer between administrations of the two compounds. Therefore, the term combination does not necessarily mean administered at the same time or as a unitary dose, but that each of the components are administered during a desired treatment period. The agents may also be administered by different routes. As is typical for chemotherapeutic regimens, a course of chemotherapy may be repeated several weeks later, and may follow the same timeframe for administration of the two compounds, or may be modified based on patient response.

In other embodiments, the pharmaceutical compositions provided herein may be provided in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

In further embodiments, the pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

In other embodiments, the pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Miccellar dosage forms can be prepared as described in U.S. Pat. No. 6,350,458.

In other embodiments, the pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, sustained, pulsed-, controlled, targeted-, and programmed-release forms.

In further embodiments, the pharmaceutical compositions provided herein may be co-formulated with other active ingredients which do not impair the desired therapeutic action, or with substances that supplement the desired action.

### Parenteral Administration

In some embodiments, the pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In other embodiments, the pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

In some embodiments, the pharmaceutical compositions provided herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthal ate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### Modified Release

In other embodiments, the pharmaceutical compositions provided herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

### 1. Matrix Controlled Release Devices

In some embodiments, the pharmaceutical compositions provided herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In one embodiment, the pharmaceutical compositions provided herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate,; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

In other embodiments, the pharmaceutical compositions provided herein in a modified release dosage form may be prepared by methods known to those skilled in the art, including direct compression, dry or wet granulation followed by compression, melt-granulation followed by compression.

### 2. Osmotic Controlled Release Devices

In some embodiments, the pharmaceutical compositions provided herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol, organic acids, such as ascorbic acid, benzoic acid, fumaric acid, citric acid, maleic acid, sebacic acid, sorbic acid, adipic acid, edetic acid, glutamic acid, p-tolunesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semi-permeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chioroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semi-permeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semi-permeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semi-permeable membrane, the composition of the core, and the number, size, and position of the delivery ports.

The pharmaceutical compositions in an osmotic controlled-release dosage form may further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In other embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

In some embodiments, the pharmaceutical compositions provided herein in a modified release dosage form may be fabricated a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates may be made by the processes know to those skilled in the art, including wet-and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### 4. Targeted Delivery

In some embodiments, the pharmaceutical compositions provided herein may also be formulated to be targeted to a particular tissue, receptor, or other area of the body of the subject to be treated, including liposome-, resealed erythrocyte-, and antibody-based delivery systems. Examples include, but are not limited to, U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874, all of which are incorporated herein by their entirety.

### Immediate Release

In some embodiments, the pharmaceutical compositions provided herein in an immediate release dosage form are capable of releasing not less than 75% of the therapeutically active ingredient or combination and/or meet the disintegration or dissolution requirements for immediate release tablets of the particular therapeutic agents or combination included in the tablet core, as set forth in USP XXII, 1990 (The United States Pharmacopeia.)

### Topical Administration

In other embodiments, the pharmaceutical compositions provided herein may be administered topically to the skin, orifices, or mucosa. The topical administration, as used herein, include (intra)dermal, conjuctival, intracomeal, intraocular, ophthalmic, auricular, transdermal, nasal, vaginal, uretheral, respiratory, and rectal administration.

In further embodiments, the pharmaceutical compositions provided herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein may also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

In some embodiments, the pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein may be provided in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions provided herein may be administered ophthalmically in the forms of solutions, suspensions, ointments, emulsions, gel-forming solutions, powders for solutions, gels, ocular inserts, and implants.

The pharmaceutical compositions provided herein may be administered intranasally or by inhalation to the respiratory tract. The pharmaceutical compositions may be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be provided as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient provided herein, a propellant as solvent; and/or an surfactant, such as sorbitan trioleate, oleic acid, or an oligolactic acid.

In another embodiment, the pharmaceutical compositions provided herein may be micronized to a size suitable for delivery by inhalation, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

In one embodiment, the pharmaceutical compositions provided herein for topical administration may be formulated to be immediate release or modified release, including delayed-, sustained-, pulsed-, controlled-, targeted, and programmed release.

### EXAMPLES

### Example 1

### Synthesis of 2-(4-ethoxyphenyl)-4-methyl-1-(4-sulfamoylphenyl)-pyrrole

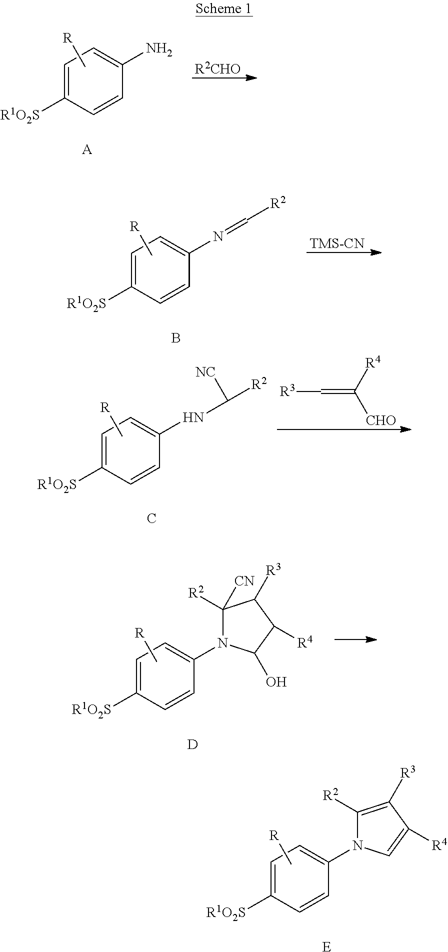

Substituted benzaldehyde undergoes dehydration condensation by reaction with aniline compound A in an inert solvent at a temperature of between 5° C. to 200° C. to give aldimine compound B. Trimethylsilyl cyanide is then reacted with aldimine compound B in the presence of a Lewis acid to afford anilinonitrile C. An α,β-unsaturated aldehyde is then reacted with anilinonitrile C to afford compound D which then undergoes dehydration and dehydrogencyanation under basic conditions in a modification of the method described in Ann. Chem. 589, 176 (1954).

### Example 2

### Synthesis of Letrozole

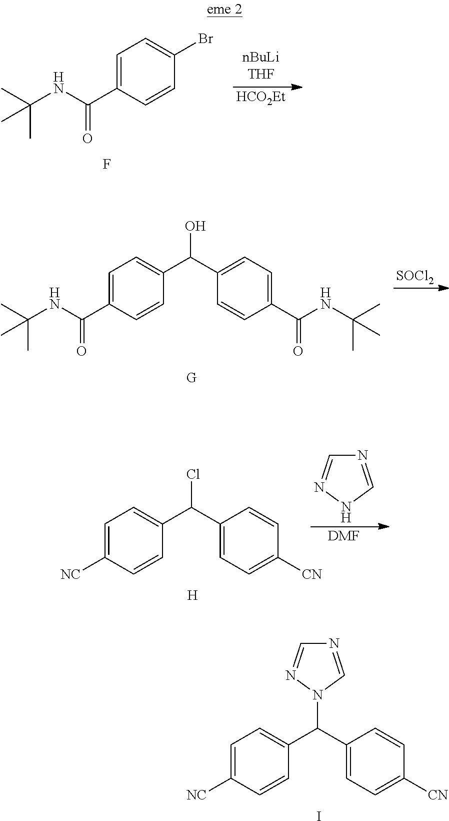

Starting amide F is treated with n-BuLi in THF at low temperature followed by ethyl formate to give the addition product G. Alcohol G is heated with thionyl chloride to afford the chloro compound H which has also been dehydrated at the amide functional groups. Treatment of bis-nitrile H with the triazole base in hot DMF will provide the desired product letrozole (1).

### Example 3

### Syntheis of Anastrozole

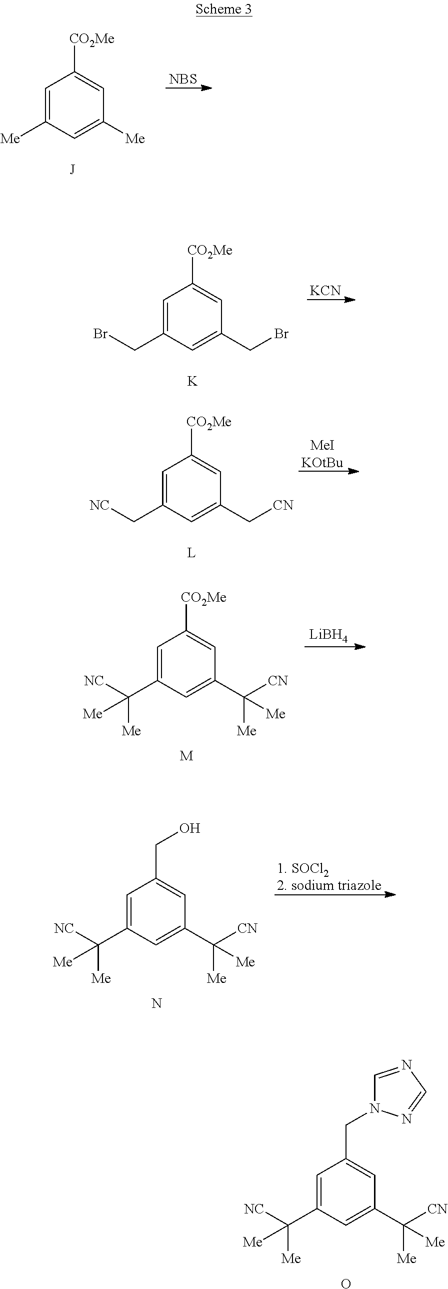

Starting ester J is brominated to give benzyl bromide K. Displacement of the bromide with potassium cyanide and alkylation of nitrile L will give nitrile M. Reduction of the ester M and conversion of the alcohol to the chloride and displacement with sodium triazole will give the final product anastrozole (O).

### Example 4

### Synthesis of Exemestane

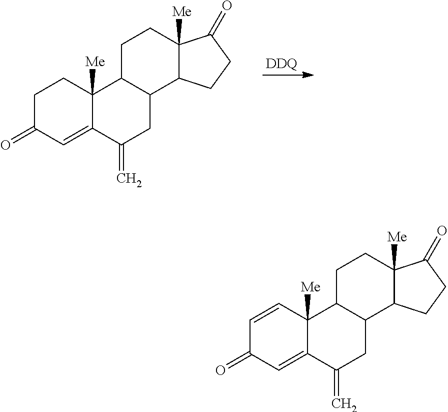

Starting material 6-methylenandrost-4-ene-3,17-dione is oxidized with DDQ to give exemestane.

### Example 5

### Pharmacokinetics and Metabolism of 2-(4-ethoxyphenyl)-4-methyl-1-(4-sulfamoylphenyl)-pyrrole

Orally administered 2-(4-ethoxyphenyl)-4-methyl-1-(4-sulfamoylphenyl)-pyrrole was rapidly absorbed in all species examined (mice, rats, dogs, and monkeys). Peak plasma concentrations were achieved between 1 and 3 hours after a dose of 5 mg/kg. The elimination half life (t_{1/2}) was 4-5 hours in rodents and dogs, and approximately 2 hours in monkeys. Oral availability was greatest in rodent, and was reduced in dogs and monkeys (59 and 34% respectively). Pharmacokinetics in human subjects demonstrated a linear dose exposure relationship from doses of 2 mg to 800 mg given orally. The half-life in human subjects is 15-18 hours.

### Example 6

### Toxicology of 2-(4-ethoxvphenyl)-4-methyl-1-(4-sulfamoylphenyl)-pyrrole

Toxicological evaluation of 2-(4-ethoxyphenyl)-4-methyl-1-(4-sulfamoylphenyl)-pyrrole in mice, rats, dogs and monkeys revealed expected findings related to inhibition of cyclooxygenase and consistent with animal safety observations with other COX-2 selective inhibitors. In single dose studies, the minimum lethal dose of 2-(4-ethoxyphenyl)-4-methyl-1-(4-sulfamoylphenyl)-pyrrole was 600 mg/kg in rats and >2000 mg/kg in dogs. An endoscopy study conducted in human subjects demonstrated no increase in gastric or duodenal toxicity compared to placebo.

### Example 7

### Biological Evaluation

### SK-BR-3 Model

Mice are injected subcutaneously in the left paw (1×10⁶ tumor cells suspended in 30% Matrigel) and tumor volume is evaluated using a phlethysmometer twice a week for 30-60 days. Implantation of human breast cancer cells (SK-BR-3) into nude mice produces tumors that will reach 0.6-2 ml between 30-50 days. Blood is drawn twice during the experiment in a 24 h protocol to assess plasma concentration and total exposure by AUC analysis. The data is expressed as the mean+/−SEM. Student's and Mann-Whitney tests are used to assess differences between means using the InStat software package.

A. Mice injected with SK-BR-3 cancer cells are treated with cytoxin i.p at doses of 50 mg/kg on days 5, 7 and 9 in the presence or absence of a composition comprising a combination of 2-(4-ethoxyphenyl)-4-methyl-1-(4-sulfamoylphenyl)-pyrrole and letrozole in the diet. The efficacies of both agents are determined by measuring tumor volume. The results from these studies may demonstrate that administration of a composition comprising a combination of 2-(4-ethoxyphenyl)-4-methyl-1-(4-sulfamoylphenyl)-pyrrole with letrozole to tumor bearing mice can delay the growth of tumors and metastasis.

B. In a second assay, mice injected with SK-BR-3 cancer cells are then treated with 5-FU on days 12 through 15. Mice injected with SK-BR-3 cancer cells are treated with 5-FU i.p at doses of 50 mg/kg on days 12, 13, 14, and 15 in the presence or absence of a composition comprising a combination of 2-(4-ethoxyphenyl)-4-methyl-1-(4-sulfamoylphenyl)-pyrrole and letrozole in the diet. The efficacy of both agents is determined by measuring tumor volume. Treatment using the composition may reduce tumor volume by up to 70%. In the same assay, 5-FU decreases tumor volume by 61%. Further, the composition and 5-FU may decrease tumor volume by 83%.

C. In a third assay, mice injected with SK-BR-3 breast cancer cells are treated with 5-FU i.p 50 mg/kg on days 14 through 17 in the presence or absence of a composition comprising a combination of 2-(4-ethoxyphenyl)-4-methyl-1-(4-sulfamoylphenyl)-pyrrole, letrozole and valdecoxib in the diet. The efficacies of both agents are determined by measuring tumor volume. Treatment with 5-FU may result in a 35% reduction in tumor volume. Treatment with the composition and valdecoxib may reduce tumor volume by 52% and 69%, respectively. In the same assay, the combination of 5-FU and the composition may decrease tumor volume by 72% while the combination of 5-FU and valdecoxib may decrease tumor volume by 74%.

### Example 8

### In Vitro Inhibition of Aromatase Activity

The in vitro inhibition of aromatase activity of the compositions of the present invention can be demonstrated as follows: A microsomal fraction is prepared from human placenta by the method as described by Thompson and Siiteri, J. Biol. Chem., Vol. 249, p. 5364 (1974). The microsomal preparation so obtained is lyophilized and stored at −40° C. The human placental microsomes are added to 1,2-³H-androstenedione and incubated for 20 minutes at 37° C. The amount of aromatization of the labelled substrate is detected by the loss of ³H₂O into the incubation medium. The substrate is removed by chloroform extraction, followed by adsorption to charcoal in suspension. The charcoal is removed by centrifugation and the steroid-free medium is counted in a liquid scintillation counter. Compositions are tested for aromatase inhibitory activity by adding them to the incubation medium prior to the addition of the microsomes. The relative cpm obtained with and without the composition is used to calculate the percent inhibition of the aromatization of androstenedione to estrone. IC₅₀ values can be determined graphically as the concentration of test composition at which the aromatization of androstenedione to estrone is reduced to 50% of control value.

### Example 9

### Pharmaceutical Comnositions and Dosage Forms Containing Letrozole

Dosage formulations comprising pharmaceutical excipients and carriers and a pharmaceutical composition comprising a combination of letrozole (A) and 2-(4-ethoxyphenyl)-4-methyl-1-(4-sulfamoylphenyl)-pyrrole (B) include:

| | Amount of A | |
|---|---|---|
| Combination | per tablet (mg) | Amount of B per tablet (mg) |
| A/B | 0.5 | 100, 200, 300, 400, 500, 600, 700, |
| | | 800, 900, 1000, 1100, 1200 |
| A/B | 1.0 | 100, 200, 300, 400, 500, 600, 700, |
| | | 800, 900, 1000, 1100, 1200 |
| A/B | 1.5 | 100, 200, 300, 400, 500, 600, 700, |
| | | 800, 900, 1000, 1100, 1200 |
| A/B | 2.0 | 100, 200, 300, 400, 500, 600, 700, |
| | | 800, 900, 1000, 1100, 1200 |
| A/B | 2.5 | 100, 200, 300, 400, 500, 600, 700, |
| | | 800, 900, 1000, 1100, 1200 |
| A/B | 3.0 | 100, 200, 300, 400, 500, 600, 700, |
| | | 800, 900, 1000, 1100, 1200 |

Dosage formulations described herein, including the formulations set forth in the above table, may be administered in a single fixed dose comprising a combination of 2-(4-ethoxyphenyl)-4-methyl-1-(4-sulfamoylphenyl)-pyrrole and letrozole or as a separate administration of a single dose of 2-(4-ethoxyphenyl)-4-methyl-1-(4-sulfamoylphenyl)-pyrrole and a single dose of letrozole.

### Example 10

### Pharmaceutical Compositions and Dosage Forms Containing Anastrozole

Dosage formulations comprising pharmaceutical excipients and carriers and a pharmaceutical composition comprising a combination of anastrozole (A) and 2-(4-ethoxyphenyl)-4-methyl-1-(4-sulfamoylphenyl)-pyrrole (B) include:

| | Amount of A | |
|---|---|---|
| Combination | per tablet (mg) | Amount of B per tablet (mg) |
| A/B | 0.2 | 100, 200, 300, 400, 500, 600, 700, |
| | | 800, 900, 1000, 1100, 1200 |
| A/B | 0.5 | 100, 200, 300, 400, 500, 600, 700, |
| | | 800, 900, 1000, 1100, 1200 |
| A/B | 0.5 | 100, 200, 300, 400, 500, 600,700, |
| | | 800, 900, 1000, 1100, 1200 |
| A/B | 0.75 | 100, 200, 300, 400, 500, 600, 700, |
| | | 800, 900, 1000, 1100, 1200 |
| A/B | 1.0 | 100, 200, 300, 400, 500, 600, 700, |
| | | 800, 900, 1000, 1100, 1200 |
| A/B | 2.0 | 100, 200, 300, 400, 500, 600, 700, |
| | | 800, 900,1000, 1100, 1200 |

Dosage formulations described herein, including the formulations set forth in the above table, may be administered in a single fixed dose comprising a combination of 2-(4-ethoxyphenyl)-4-methyl-1-(4-sulfamoylphenyl)-pyrrole and anastrozole or as a separate administration of a single dose of 2-(4-ethoxyphenyl)-4-methyl-1-(4-sulfamoylphenyl)-pyrrole and a single dose of anastrozole.

### Example 11

### Pharmaceutical Compositions and Dosage Forms Containing Exemestane

Dosage formulations comprising pharmaceutical excipients and carriers and a pharmaceutical composition comprising a combination of exemestane (A) and 2-(4-ethoxyphenyl)-4-methyl-1-(4-sulfamoylphenyl)-pyrrole (B) include:

| | Amount of A | |
|---|---|---|
| Combination | (per tablet mg) | Amount of B per tablet (mg) |
| A/B | 5.0 | 100, 200, 300, 400, 500, 600, 700, |
| | | 800, 900, 1000, 1100, 1200 |
| A/B | 10.0 | 100, 200, 300, 400, 500, 600, 700, |
| | | 800, 900, 1000, 1100, 1200 |
| A/B | 15.0 | 100, 200, 300, 400, 500, 600, 700, |
| | | 800, 900, 1000, 1100, 1200 |
| A/B | 20.0 | 100, 200, 300, 400, 500, 600, 700, |
| | | 800, 900, 1000, 1100, 1200 |
| A/B | 25.0 | 100, 200, 300, 400, 500, 600, 700, |
| | | 800, 900, 1000, 1100, 1200 |
| A/B | 35.0 | 100, 200, 300, 400, 500, 600, 700, |
| | | 800, 900, 1000, 1100, 1200 |

Dosage formulations described herein, including the formulations set forth in the above table, may be administered in a single fixed dose comprising a combination of 2-(4-ethoxyphenyl)-4-methyl-1-(4-sulfamoylphenyl)-pyrrole and exemestane or as a separate administration of a single dose of 2-(4-ethoxyphenyl)-4-methyl-1-(4-sulfamoylphenyl)-pyrrole and a single dose of exemestane.

### Example 12

### Pharmaceutical Compositions and Dosage Forms Containing Toremifene

Dosage formulations comprising pharmaceutical excipients and carriers and a pharmaceutical composition comprising a combination of toremifene (A) and 2-(4-ethoxyphenyl)-4-methyl-1-(4-sulfamoylphenyl)-pyrrole (B) include:

| | Amount of A | |
|---|---|---|
| Combination | per tablet (mg) | Amount of B per tablet (mg) |
| A/B | 25 | 100, 200, 300, 400, 500, 600, 700, |
| | | 800, 900, 1000, 1100, 1200 |
| A/B | 35 | 100, 200, 300, 400, 500, 600, 700, |
| | | 800, 900, 1000, 1100, 1200 |
| A/B | 45 | 100, 200, 300, 400, 500, 600, 700, |
| | | 800, 900, 1000, 1100, 1200 |
| A/B | 55 | 100, 200, 300, 400, 500, 600, 700, |
| | | 800, 900, 1000, 1100, 1200 |
| A/B | 65 | 100, 200, 300, 400, 500, 600, 700, |
| | | 800, 900, 1000, 1100, 1200 |
| A/B | 75 | 100, 200, 300, 400, 500, 600, 700, |
| | | 800, 900,1000, 1100, 1200 |

Dosage formulations described herein, including the formulations set forth in the above table, may be administered in a single fixed dose comprising a combination of 2-(4-ethoxyphenyl)-4-methyl-1-(4-sulfamoylphenyl)-pyrrole and toremifene or as a separate administration of a single dose of 2-(4-ethoxyphenyl)-4-methyl-1-(4-sulfamoylphenyl)-pyrrole and a single dose of toremifene.

### Example 13

### Pharmaceutical Compositions and Dosage Forms Containing Tamoxifen

Dosage formulations comprising pharmaceutical excipients and carriers and a pharmaceutical composition comprising a combination of tamoxifen (A) and 2-(4-ethoxyphenyl)-4-methyl-1-(4-sulfamoylphenyl)-pyrrole (B) include:

| | Amount of A | |
|---|---|---|
| Combination | per tablet (mg) | Amount of B per tablet (mg) |
| A/B | 10 | 100, 200, 300, 400, 500, 600, 700, |
| | | 800, 900, 1000, 1100, 1200 |
| A/B | 20 | 100, 200, 300, 400, 500, 600, 700, |
| | | 800, 900, 1000, 1100, 1200 |
| A/B | 30 | 100, 200, 300, 400, 500, 600, 700, |
| | | 800, 900, 1000, 1100, 1200 |
| A/B | 40 | 100, 200, 300, 400, 500, 600, 700, |
| | | 800, 900, 1000, 1100, 1200 |
| A/B | 50 | 100, 200, 300, 400, 500, 600, 700, |
| | | 800, 900, 1000, 1100, 1200 |
| A/B | 60 | 100, 200, 300, 400, 500, 600, 700, |
| | | 800, 900, 1000, 1100, 1200 |

Dosage formulations described herein, including the formulations set forth in the above table, may be administered in a single fixed dose comprising a combination of 2-(4-ethoxyphenyl)-4-methyl-1-(4-sulfamoylphenyl)-pyrrole and tamoxifen or as a separate administration of a single dose of 2-(4-ethoxyphenyl)-4-methyl-1-(4-sulfamoylphenyl)-pyrrole and a single dose of tamoxifen.

1. A method for treating a subject having cancer, comprising administering to the subject, a therapeutically effective amount of a combination comprising a 1,2-diphenylpyrrole derivative and an aromatase inhibitor or their respective pharmaceutically acceptable salts, solvates, polymorphs or prodrugs.

2. The method of claim 1 wherein the 1,2-diphenylpyrrole derivative has the following formula: 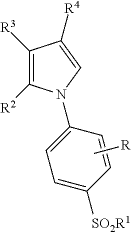
wherein:
R is a hydrogen atom, a halogen atom or an alkyl group having from 1 to 6 carbon atoms;
R¹ is an alkyl group having from 1 to 6 carbon atoms or an amino group;
R² is a phenyl group which is unsubstituted or is substituted by at least one substituent selected from the group consisting of substituents a and substituents β;
R³ is a hydrogen atom, a halogen atom or an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms;
R⁴ is a hydrogen atom; an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms; a cycloalkyl group having from 3 to 8 carbon atoms, an aryl group; or an aralkyl group; said aryl group having from 6 to 14 ring carbon atoms in a carbocyclic ring and are unsubstituted or are substituted by at least one substituent selected from the group consisting of substituents a and substituents β;
said aralkyl group are an alkyl group having from 1 to 6 carbon atoms and which are substituted by at least one aryl group as defined above;
said substituents a are selected from the group consisting of a hydroxy group, a halogen atom, an alkoxy group having from 1 to 6 carbon atoms and an alkylthio group having from 1 to 6 carbon atoms;
said substituents β are selected from the group consisting of an alkyl group which has from 1 to 6 carbon atoms and which is unsubstituted or are substituted by at least one substituent selected from the group consisting of a hydroxy group, a halogen atom, an alkoxy group having from 1 to 6 carbon atoms and an alkylthio group having from 1 to 6 carbon atoms; an alkanoyloxy group having from 1 to 6 carbon atoms; a mercapto group; an alkanoylthio group having from 1 to 6 carbon atoms; an alkylsulfinyl group having from 1 to 6 carbon atoms; a cycloalkloxy group having from 3 to 8 carbon atoms; a haloalkoxy group having from 1 to 6 carbon atoms; and an alkylenedioxy group having from 1 to 6 carbon atoms; or a pharmaceutically acceptable salt, solvate, or prodrug.

3. The method of claim 2 wherein:
R is a hydrogen atom, a halogen atom or an alkyl group having from 1 to 4 carbon atoms;
R¹ is a methyl group or an amino group;
R² is an unsubstituted phenyl group or a phenyl group which is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and which is substituted by at least one substituent selected from the group consisting of a halogen atom, an alkoxy group having from 1 to 4 carbon atoms and an alkylthio group having from 1 to 4 carbon atoms; a haloalkoxy group having from 1 to 4 carbon atoms; and an alkylenedioxy group having from 1 to 4 carbon atoms;
R³ is a hydrogen atom, a halogen atom, an unsubstituted alkyl group having from 1 to 4 carbon atoms or a substituted alkyl group having from 1 to 4 carbon atoms and substituted by at least one substituent selected from the group consisting of a halogen atom, an alkoxy group having from 1 to 4 carbon atoms and an alkylthio group having from 1 to 4 carbon atoms;
R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to carbon atoms; a cycloalkyl group having from 3 to 6 carbon atoms; an aryl group which has from 6 to 10 ring carbon atoms and which is unsubstituted or is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to 4 carbon atoms; and a cycloalkyloxy group having from 3 to 7 carbon atoms; an aralkyl group having from 1 to 4 carbon atoms in the alkyl part and containing at least one said aryl group; or a pharmaceutically acceptable salt, solvate, or prodrug.

4. The method of claim 3 wherein:
R is a hydrogen atom;
R¹ is an amino group;
R² is an unsubstituted phenyl group or a phenyl group which is substituted by at least one substituent selected from the group consisting of a halogen atom, an alkoxy group having from 1 to 4 carbon atoms, an alkylthio group having from 1 to 4 carbon atoms, an alkyl group having from 1 to 4 carbon atoms, a haloalkyl group having from 1 to 4 carbon atoms, a haloalkoxy group having from 1 to 4 carbon atoms and a alkylenedioxy group having from 1 to 4 carbon atoms;
R³ is a hydrogen atom, a halogen atom, an alkyl group having from 1 to 4 carbon atoms or a haloalkyl group having from 1 to 4 carbon atoms;
R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group and an alkoxy group having from 1 to 4 carbon atoms; a cycloalkyl group having from 3 to 6 carbon atoms; an aryl group which has from 6 to 10 ring carbon atoms and which is unsubstituted or is substituted by at least one substituent selected from the group consisting of a hydroxy group; a halogen atom; an alkoxy group having from 1 to 4 carbon atoms; an unsubstituted alkyl group having from 1 to 4 carbon atoms; an alkyl group having from 1 to 4 carbon atoms and which is unsubstituted or substituted by at least one halogen atom; and a cycloalkyloxy group having from 3 to 7 carbon atoms; and an aralkyl group having from 1 to 4 carbon atoms in the alkyl part and containing at least one said aryl group; or a pharmaceutically acceptable salt, solvate, or prodrug.

5. The method of claim 4 wherein the 1,2-diphenylpyrrole derivative is selected from the group consisting of: 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole, 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole.

6. The method of claim 5 wherein the 1,2-diphenylpyrrole derivative is 2-(4-ethoxyphenyl)-4-methyl-1-(4-sulfamoylphenyl)-pyrrole.

7. The method of claim 1 wherein the aromatase inhibitor has the following formula 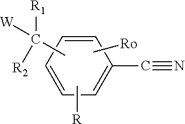
wherein R and Rₒ represent hydrogen or lower alkyl; or R and Rₒ located on adjacent carbon atoms and together when combined with the benzene ring to which they are attached form a naphthalene or tetrahydronaphthalene ring; R₁ represents hydrogen; R₂ represents hydrogen, lower alkyl, (lower alkyl, aryl or aryl-lower alkyl)-thio, lower alkenyl, aryl, aryl-lower alkyl, C₃-C₆-cycloalkyl, or C₃-C₆-cycloalkyl-lower alkyl; or R₁ and R₂ combined represent lower alkylidene, mono- or di-aryl-lower alkylidene; R₁ and R₂ combined also represent C₄-C₆-straight chain alkylene, lower alkyl-substituted straight chain alkylene or CH₂-ortho-phenylene-CH₂; W represents 1-(1,2,4- or 1,3,4))-triazolyl or 1-(1,2,4 or 1,3,4-triazolyl substituted by lower alkyl; aryl within the above definitions represents phenyl or phenyl substituted by one or two substituents selected from lower alkyl, lower alkoxy, hydroxy, lower alkanoyloxy, aroyloxy, nitro, amino, halogen, trifluoromethyl, cyano, carboxy, carboxy funtionalized in form of a pharmaceutically acceptable ester or amide, lower alkanoyl, aroyl, lower alkylsulfonyl, sulfamoyl, N-lower alkylsulfamoyl or N,N-di-lower alkylsulfamoyl; and aryl within the above definitions also represents 2-, 3-, or 4-pyridyl or a said heterocyclic radical monosubstituted by lower alkyl, lower alkoxy, cyano or halogen; and aroyl within the above definitions represents benzoyl or benzoyl substituted by lower alkyl, lower alkoxy, halogen or trifluoromethyl; or a pharmaceutically acceptable salt thereof

8. The method of claim 7 wherein the aromatase inhibitor has the following formula 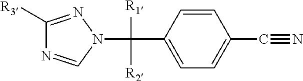
R_{1′} and R_{3′} represent hydrogen; R_{2′} represents 3-pyridyl, p-cyanobenzyl or p-cyanophenyl; or a pharmaceutically acceptable salt thereof.

9. The method of claim 8 wherein the aromatase inhibitor is letrozole.

10. The method of claim 1 wherein the 1,2-diphenylpyrrole derivative is 2-(4-ethoxyphenyl)-4-methyl-1-(4-sulfamoylphenyl)-pyrrole and the aromatase inhibitor is letrozole.

11. The method of claim 1 wherein the aromatase inhibitor has the following formula 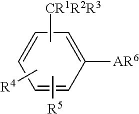
wherein R¹ is an azido, carbamoyl, cyano, formyl, hydroxy or nitro radical, a 1-6C 1-hydroxyalkyl, alkoxy, alkylcarbamoyl, alkylthio, alkylsulphinyl or alkylsulphonyl radical, a 2-cyanoethyl radical, optionally bearing one to four 1-6C alkyl substituents, or a 2-6C alkanoyl, halogenoalkanoyl, alkanoyloxy, alkanoylamino, dialkylcarbamoyl or alkoxycarbonyl radical; R² and R³, which may be the same or different, are each a 1-6C alkyl, deuterioalkyl or halogenoalkyl radical, or R¹, R², R³ C— is a 1,1-dicyanoethyl or trifluoromethylsulphonyl radical; R⁴ is a hydrogen or halogen atom, a cyano or nitro radical or a 1-6C alkyl or halogenoalkyl radical; R⁵ has any of the values defined above for the group R¹, R², R³ C, or has any of the values defined above for R⁴, or is a carbamoyl, 1-pyrrolidinyl-carbonyl, piperidinocarbonyl, morpholinocarbonyl or nitro radical, a 1-6C alkoxy or halogenoalkoxy radical or a 2 6C alkanoyl or alkoxy-carbonyl radical; A is a methylene or ethylene radical optionally bearing one or more substituents selected from the group consisting of deuterium and halogen atoms, carbamoyl, cyano and hydroxy radicals, 1-6C alkyl and alkoxy radicals, and 2 6C alkanoyloxy radicals provided that when A is linked to R⁶ through a nitrogen atom thereof, it may not bear a hydroxy, alkoxy or alkanoyloxy substituent on the carbon atom adjacent to such nitrogen atoms; and R⁶is a 1H-1,2,4-triazol-1-yl or 4H-1,2,4-triazol-4 yl; and the pharmaceutically acceptable acid addition salts thereof.

12. The method of claim 11 wherein the aromatase inhibitor is anastrozole.

13. The method of claim 1 wherein the 1,2-diphenylpyrrole derivative is 2-(4-ethoxyphenyl)-4-methyl-1-(4-sulfamoylphenyl)-pyrrole and the aromatase inhibitor is anastrozole.

14. The method of claim 1 wherein the aromatase inhibitor is exemestane.

15. The method of claim 1 wherein the 1,2-diphenylpyrrole derivative is 2-(4-ethoxyphenyl)-4-methyl-1-(4-sulfamoylphenyl)-pyrrole and the aromatase inhibitor is exemestane.

16. The method of claim 1 wherein the 1,2-diphenylpyrrole derivative and the aromatase inhibitor are administered sequentially in either order or simultaneously.

17. The method of claim 1 wherein the 1,2-diphenylpyrrole derivative is administered first.

18. The method of claim 1 wherein the aromatase inhibitor is administered first.

19. The method of claim 1 wherein administering the combination enhances treatment of the subject.

20. The method of claim 1 wherein administering the combination reduces the side effects of the treatment of cancer compared to a treatment with the aromatase inhibitor alone or a treatment with the 1,2-diphenylpyrrole derivative alone.

21.-268. (canceled)
